SEQUENCE LISTING (9 sequences)

SEQ ID NO: 1 - DNA, 169 bp, synthetic construct
  source 1..169: mol_type = other DNA
tgcgcacccttagcgagaggtttatcattaaggtcaacctctggatgttgtttcggcatcctgcattgaatctgagttactgtctgttttccttgttggaacggagagcatcgcctgatgctctccgagccaaccaggaaacccgttttttctgacgtaagggtgcgca

SEQ ID NO: 2 - DNA, 963 bp, synthetic construct
  source 1..963: mol_type = other DNA
atgaaatctgcagagtatctgaatacgttccgccttaggaatttgggcctccccgtgatgaacaatctccacgatatgagcaaggcgactcgaatatccgtggaaacgctgagactgctcatctatacagcagactttcggtacaggatctacacggtcgaaaagaaggggcctgagaaacgcatgcgaacaatttatcaacctagccgagagctcaaggcgttgcagggctgggttcttcgaaacatccttgacaaactctcatcatcaccctttagtattgggtttgaaaagcaccaaagcatccttaacaacgcgacgccacacataggtgccaatttcatattgaacatcgacttggaggatttttttccgagcctcacagccaataaagtgttcggtgtttttcacagtcttgggtacaatcgccttattagttccgttcttaccaagatttgttgttacaagaatctcttgccccagggagcacccagcagtccgaaattggcgaatttgatttgttccaagctcgattatcgaatacaagggtacgcgggcagccggggactcatctatacccgctacgcagacgatcttacgctgtctgcccaatcaatgaagaaggtcgtaaaggcgcgggatttcttgttttctatcatcccgtccgagggcttggtaattaattccaaaaagacttgtatctcaggaccacgatctcagcgaaaagtgacaggactcgtcatttctcaagaaaaagtcggtatagggagagagaagtataaggaaatccgcgcgaagatccaccacatattctgtggcaagagcagcgagatagaacacgtccgaggctggttgtccttcatactgagcgtggactcaaaaagccaccgccggttgatcacctatatttcaaaactggaaaagaaatatggaaagaacccactcaacaaagctaaaacatag

SEQ ID NO: 3 - DNA, 1761 bp, synthetic construct
  source 1..1761: mol_type = other DNA
atgacaaaaacttcaaagctggatgcgctgcgggcggctactagtagggaagatttggcgaagattctcgacataaagttggtgtttctgacaaacgtgttgtaccgcataggatccgacaaccagtatacgcaattcacaatacccaaaaagggtaaaggtgtccgcaccatcagcgcaccaacggaccgacttaaggatatacagaggaggatttgtgatcttcttagtgactgtagggatgaaatctttgcgattaggaagatctctaataattactcattcggcttcgaaagaggaaaatcaattatactcaatgcttacaagcatcgagggaagcaaattatattgaacatcgaccttaaggacttctttgagagctttaactttgggagagtccgggggtactttctctccaaccaggacttcttgttgaacccagttgtggcaacaacgttggcgaaggccgcctgctacaacgggactctgcctcaggggtccccatgttcccctattataagtaaccttatctgtaacattatggacatgcggctcgcaaagctcgccaagaagtacggctgcacttatagtcgatatgcggatgacattacgatcagcaccaataaaaataccttcccgttggagatggcgactgtgcagcctgaaggggttgtgctgggcaaagtgctcgtaaaggagattgaaaattcaggtttcgagattaacgattctaagactagattgacctacaaaacaagtaggcaagaagtcaccgggctgacggttaatcggattgtaaacattgatcggtgctactacaaaaagacgagggcgctggctcacgcattgtatcggacaggagaatataaggtcccagacgagaacggtgttctggtatctggagggcttgacaagttggagggtatgtttgggtttatcgaccaggtggataaattcaacaacattaaaaaaaagttgaataagcaacccgacagatatgttctgacaaatgccactttgcacggatttaagctcaaattgaacgccagggagaaagcctatagcaaattcatctactacaaattcttccacggtaatacttgtcccacgatcataacagagggtaagacggataggatttaccttaaagctgccctccatagcctcgagacaagttatcctgaactgtttcgggagaaaacagatagtaagaagaaggagataaatctgaatatttttaaaagcaatgagaagaccaagtatttcctggatctcagcggcggcacagcagacctcaagaaattcgtggaacgctacaaaaataactacgcttcctattacggcagcgtaccgaaacaaccggtgataatggtgcttgataacgacacaggcccgtcagacctgttgaactttttgagaaacaaagttaagagttgtccagatgatgtaacagaaatgcgcaagatgaagtacatacatgtgttttacaatctgtacatagttctgactcccctgtctccatctggagagcaaacgtctatggaggacctctttcctaaagatatattggacattaagatagatggcaagaaattcaataaaaacaatgacggtgactccaaaacagagtatgggaagcacatattctcaatgcgcgttgtacgagataaaaagaggaagatagatttcaaggcattttgctgtatcttcgatgctattaaggatattaaagaacattacaaactgatgttgaattcctag

SEQ ID NO: 4 - protein, 319 aa, synthetic construct
KSAEYLNTFRLRNLGLPVMNNLHDMSKATRISVETLRLLIYTADFRYRIYTVEKKGPEKRMRTIYQPSRELKALQGWVLRNILDKLSSSPFSIGFEKHQSILNNATPHIGANFILNIDLEDFFPSLTANKVFGVFHSLGYNRLISSVLTKICCYKNLLPQGAPSSPKLANLICSKLDYRIQGYAGSRGLIYTRYADDLTLSAQSMKKVVKARDFLFSIIPSEGLVINSKKTCISGPRSQRKVTGLVISQEKVGIGREKYKEIRAKIHHIFCGKSSEIEHVRGWLSFILSVDSKSHRRLITYISKLEKKYGKNPLNKAKT

SEQ ID NO: 5 - protein, 586 aa, synthetic construct
MTKTSKLDALRAATSREDLAKILDIKLVFLTNVLYRIGSDNQYTQFTIPKKGKGVRTISAPTDRLKDIQRRICDLLSDCRDEIFAIRKISNNYSFGFERGKSIILNAYKHRGKQIILNIDLKDFFESFNFGRVRGYFLSNQDFLLNPVVATTLAKAACYNGTLPQGSPCSPIISNLICNIMDMRLAKLAKKYGCTYSRYADDITISTNKNTFPLEMATVQPEGVVLGKVLVKEIENSGFEINDSKTRLTYKTSRQEVTGLTVNRIVNIDRCYYKKTRALAHALYRTGEYKVPDENGVLVSGGLDKLEGMFGFIDQVDKFNNIKKKLNKQPDRYVLTNATLHGFKLKLNAREKAYSKFIYYKFFHGNTCPTIITEGKTDRIYLKAALHSLETSYPELFREKTDSKKKEINLNIFKSNEKTKYFLDLSGGTADLKKFVERYKNNYASYYGSVPKQPVIMVLDNDTGPSDLLNFLRNKVKSCPDDVTEMRKMKYIHVFYNLYIVLTPLSPSGEQTSMEDLFPKDILDIKIDGKKFNKNNDGDSKTEYGKHIFSMRVVRDKKRKIDFKAFCCIFDAIKDIKEHYKLMLNS

SEQ ID NO: 6 - protein, 312 aa, synthetic construct
MSIDIETTLQKAYPDFDVLLKSRPATHYKVYKIPKRTIGYRIIAQPTPRVKAIQRDIIEILKQHTHIHDAATAYVDGKNILDNAKIHQSSVYLLKLDLVNFFNKITPELLFKALARQKVDISDTNKNLLKQFCFWNRTKRKNGALVLSVGAPSSPFISNIVMSSFDEEISSFCKENKISYSRYADDLTFSTNERDVLGLAHQKVKTTLIRFFGTRIIINNNKIVYSSKAHNRHVTGVTLTNNNKLSLGRERKRYITSLVFKFKEGKLSNVDINHLRGLIGFAYNIEPAFIERLEKKYGESTIKSIKKYSEGG

SEQ ID NO: 7 - protein, 311 aa, synthetic construct
MDILQHISDLLLTKKSEIISFSLTAPYRYKIYKIAKRNSDKKRTIAHPSKELKFIQREITEYLTDKLPVHECAFAYKKGSSIKTNAQVHLHTKYLLKMDFENFFPSITPRLFFSKLRLANIDLTADDKVLLENILFFKSKRNSNLRLSIGAPSSPLISNFVMYFWDIEVQEICSKIGVNYTRYADDLTFSTNNKDVLFDIPDMLENVLPKYSLGRIRINHEKTVFSSKGHNRHVTGITLTNDNKLSIGRERKRKISAMIHHFINGKLSTDECNKLVGLLAFAKNIEPSFYKSMVIKYGSDNIYKLQKQKDK

SEQ ID NO: 8 - DNA, 169 bp, synthetic construct; identical to SEQ ID NO: 1
  source 1..169: mol_type = other DNA

SEQ ID NO: 9 - DNA, 199 bp, synthetic construct
  source 1..199: mol_type = other DNA
tgataagattccgtatgcgcacccttagcgagaggtttatcattaaggtcaacctctggatgttgtttcggcatcctgcattgaatctgagttactgtctgttttccttgttggaacggagagcatcgcctgatgctctccgagccaaccaggaaacccgttttttctgacgtaagggtgcgcatacggaatcttatca

(19) United States
(12) Patent Application Publication
(10) Pub. No.: US 2025/0243516 A1
(43) Pub. Date: Jul. 31, 2025

(54) Dual Cut Retron Editors for Genomic Insertions and Deletions

(71) Applicants: The J. David Gladstone Institutes, a testamentary trust established under the Will of J. David, San Francisco, CA (US); The Regents of the University of California, Oakland, CA (US)

(72) Inventors: Seth Shipman, San Francisco, CA (US); Santiago C. Lopez, Oakland, CA (US)

(21) Appl. No.: 19/105,747

(22) PCT Filed: Aug. 24, 2023
(86) PCT No.: PCT/US2023/072799

§ 371 Date: Feb. 21, 2025

Related U.S. Application Data
(60) Provisional application No. 63/400,434, filed on Aug. 24, 2022.

(51) Int. Cl. C12N 15/90; C12N 9/12; C12N 9/22; C12N 15/11

(52) U.S. Cl. CPC C12N 15/907; C12N 9/1276; C12N 9/22; C12N 15/11; C12N 2310/20; C12N 2800/80

(57) ABSTRACT

Described herein are retron-related constructs, expression systems, and methods for precisely deleting, inserting and/or replacing genomic DNA within cells. The inventors have found that CRISPR “single-cutter” methods are substantially less efficient at insertion/replacement of large fragments in comparison to the dual-cutter constructs, expression systems, and methods described herein.

### CROSS-REFERENCE TO RELATED APPLICATION

This application claims the benefit of priority of U.S. Provisional Application No. 63/400,434, filed Aug. 24, 2022, the contents of which are incorporated by reference herein in its entirety for any purpose.

### SEQUENCE LISTING

This application contains a Sequence Listing which has been submitted electronically in ST26 format and is hereby incorporated by reference in its entirety. Said ST26 file, created on Aug. 23, 2023, is named “3730214WO1.xml” and is 13,627 bytes in size.

### BACKGROUND

CRISPR/Cas9 system is a flexible and fast developing technology that has been extensively used to make mutations in all kinds of organisms. CRISPR nucleases have been used with exogenously delivered DNA repair templates, to induce gene-sized modifications (insertions or deletions), for example, in yeast genomes using homologous recombination. However, to date most CRISPR-related mutation/repair methods have involved small insertions/deletions and such methods lack the ability to generate DNA repair templates in situ. Moreover, currently available CRISPR-related mutation/repair methods typically rely on a “single-cutter” approach, where the CRISPR nuclease is targeted by a single guide RNA to a single site in the genome in hopes that a donor DNA fragment can be integrated into the genomic locus that was cleaved just once by the CRISPR nuclease.

### SUMMARY

Described herein are constructs, expression systems, and methods for precisely deleting, inserting and/or replacing genomic DNA within cells. The inventors have found that CRISPR “single-cutter” methods are substantially less efficient at insertion of large fragments in comparison to the dual-cutter methods described herein. Such CRISPR “single-cutter” methods are also more restricted in terms of the potential range of genomic alterations that can be obtained.

The methods involve cutting a cell's genomic DNA at two sites targeted by two separate guide RNAs, each encoded by a single retron non-coding RNA (ncRNA) expressed in the cell. The ncRNA can also include homology arms, one homology arm being complementary to sequence adjacent to the target site of the first guide RNA, and a second homology arm being complementary to sequence adjacent to the target site of the second guide RNA. A portion of the ncRNA can also encode a donor DNA that can be reverse transcribed within the cell to provide the donor DNA. The reverse transcribed donor DNA can be flanked by the first and second homology arms. Donor DNA is thus synthesized within the cell, providing enhanced amounts of DNA compared to transfection or other procedures.

As shown herein, the orientation of the donor DNA, the homology arms, and the guide RNAs can significantly affect the efficiency of genomic editing. In some cases improved editing efficiency can be obtained by using guide RNAs can bind to the strand that is initially replaced by the single stranded reverse-transcribed donor DNA. When the guide RNAs bind to the strand that donor DNA does not initially replace, the editing frequency can in some cases be significantly reduced.

Editing retrons (editrons) are therefore described herein that include a modified retron non-coding RNA (ncRNA) having a sequence for a first guide RNA, a sequence for a second guide RNA, a first RNA template for a first homology arm DNA, and a second RNA template for a second homology arm DNA. The first homology arm DNA and the second homology arm DNA are, for example, separately complementary to distinct sites of a target genomic site. The editing retron (editron) can further include an RNA template for a donor DNA that can be reverse transcribed by a reverse transcriptase. The donor DNA can replace a portion of a target gene, or a flanking region of a gene. The first RNA template for a first homology arm DNA can flank one end of a template for a donor DNA and the second RNA template for a second homology arm DNA can flank the second end of the template for the donor DNA. The RNA template for a donor DNA can also include an initiation site for a reverse transcriptase.

Also described herein are expression systems that are useful for expressing editing retrons (editrons) and method for genomic editing that employ the editing retrons (editrons).

One aspect provides an editing retron (editron) comprising a modified retron non-coding RNA (ncRNA) comprising a sequence for a first guide RNA, a sequence for a second guide RNA, a first RNA template for a first homology arm DNA, and a second RNA template for a second homology arm DNA. In one aspect, the first homology arm DNA and the second homology arm DNA are separately complementary to distinct sites of a target genomic site. One aspect provides an RNA template for a donor DNA that can be reverse transcribed by a reverse transcriptase. In one aspect, the reverse transcribed donor DNA is single stranded. In one aspect, the first RNA template for a first homology arm DNA flanks one end of a template for a donor DNA and the second RNA template for a second homology arm DNA flanks the second end of the template for the donor DNA. In one aspect, the first guide RNA and the second guide RNA bind to a target strand that is replaced by the reverse transcribed donor DNA. In one aspect, RNA template for a donor DNA comprises an initiation site for a reverse transcriptase.

One aspect provides an expression system comprising at least one expression cassette comprising a promoter operably linked to coding region for an editing retron (editron) comprising a modified retron non-coding RNA (ncRNA) comprising a sequence for a first guide RNA, a sequence for a second guide RNA, a first RNA template for a first homology arm DNA, and a second RNA template for a second homology arm DNA. One aspect provides at least one expression cassette comprising a promoter operably linked to a coding region for a reverse transcriptase. One aspect provides at least one expression cassette comprising a promoter operably linked to a coding region for a cas nuclease. In one aspect, the first homology arm DNA and the second homology arm DNA are separately complementary to distinct sites of a target genomic site. In one aspect, the editing retron (editron) further comprises an RNA template for a donor DNA that can be reverse transcribed by a reverse transcriptase. In one aspect, the reverse transcribed donor DNA is single stranded. In one aspect, the first RNA template for a first homology arm DNA flanks one end of a template for a donor DNA and the second RNA template for a second homology arm DNA flanks the second end of the template for the donor DNA. In one aspect, the first guide RNA and the second guide RNA bind to a target strand that is replaced by the reverse transcribed donor DNA. In one aspect, the RNA template for a donor DNA comprises an initiation site for a reverse transcriptase.

One aspect provides a method comprising: (a) transforming a population of host cells, each host cell comprising a reverse transcriptase and a cas nuclease, with an expression system comprising at least one expression cassette comprising a promoter operably linked to coding region for an editing retron (editron) comprising a modified retron non-coding RNA (ncRNA) comprising a sequence for a first guide RNA, a sequence for a second guide RNA, a first RNA template for a first homology arm DNA, and a second RNA template for a second homology arm DNA. In one aspect, the first homology arm DNA and the second homology arm DNA are separately complementary to distinct sites of a target genomic site. In one aspect, the editing retron (editron) further comprises an RNA template for a donor DNA that can be reverse transcribed by a reverse transcriptase. In one aspect, the reverse transcribed donor DNA is single stranded. In one aspect, the first RNA template for a first homology arm DNA flanks one end of a template for a donor DNA and the second RNA template for a second homology arm DNA flanks the second end of the template for the donor DNA. In one aspect, the first guide RNA and the second guide RNA bind to a target strand that is replaced by the reverse transcribed donor DNA. In one aspect, the RNA template for a donor DNA comprises an initiation site for a reverse transcriptase. One aspect provides at least 2-fold, at least 3-fold, at least 4-fold, at least 5-fold, at least 7-fold, at least 10-fold, at least 15-fold, at least 20-fold, at least 25-fold, at least 30-fold, at least 40-fold, at least 50-fold, at least 60-fold, at least 70-fold, at least 80-fold, at least 90-fold, at least 100-fold, or at least 200-fold more genomic deletions, genomic replacements, and/or genomic insertions compared to use of a single cutter guide RNA.

### DESCRIPTION OF THE FIGURES

FIG. 1A-1C illustrate a wild type retron structure compared to retrons modified as described herein to provide guide RNAs and donor DNAs for genetic modification. FIG. 1A is a schematic diagram of a wild type retron, showing the DNA portion of the retron in blue and the RNA portion of the retron in pink. An expression cassette is shown at the bottom that encodes a non-coding RNA (ncRNA) for the retron show above. The ncRNA provides a template for reverse transcription of the DNA portion. FIG. 1B a schematic diagram of a single guide ‘editron’—a retron modified to supply a single guide RNA (gRNA, dark pink, linked to the ncRNA via its 3′ end) and a donor DNA (red) for insertion of the donor DNA after a single cut. FIG. 1C a schematic diagram of a double guide ‘editron’—a retron modified to supply two guide RNAs (gRNAs, dark pink) and a donor DNA (red). The 3′ gRNA is linked at the 3′ end to the ncRNA while the 5′ gRNA is linked at the 5′ end to the ncRNA. The double guide ‘editron’ can cut the genome in two different target locations.

FIG. 2A-2B illustrate deletion of a yeast ADE2 gene using double guide ‘editrons.’ FIG. 2A shows the ADE2 genomic locus with the positioning and orientation of 3′ gRNAs and 5′ gRNAs for four different experimental constructs. The gRNA sequences are shown in red. In constructs 364 and 366 the 5′ gRNA (5′ referring to the position in the ncRNA expression cassette) targets the 5′ end of the ADE2 gene and the 3′ gRNA targets the 3′ end of the ADE2 gene. In constructs 365 and 367 the 5′ gRNA targets the 3′ end of the ADE2 gene and the 3′ gRNA targets the 5′ end of the ADE2 gene. For constructs 364 and 365, the reverse-transcribed donor DNA (orange) is produced in the reverse direction relative to the ADE2 gene and gRNA orientation. In other words, the guide RNAs for the constructs 364 and 365 bind to the target strand that is initially replaced by the donor DNA. In contrast, for constructs 366 and 367 the reverse-transcribed donor is produced in the same orientation as the ADE2 gene and gRNAs, and hence the guide RNAs were designed to bind the strand that is not initially replaced by the donor DNA. FIG. 2B shows deleted and undeleted ADE2 genomic fragments detected by polymerase chain reaction (PCR) amplification of the ADE2 genomic locus after cleavage by cas nuclease complexes with the different 364, 365, 366, and 367 editron gRNAs. The deleted ADE2 fragments were smaller and traveled further through the polyacrylamide gel used for fragment separation than the undeleted wild type ADE2 fragments. As illustrated, the 364 and 365 editrons provide improved deletion of the ADE2 locus relative to the 366 and 367 editrons.

FIG. 3A-3B illustrate improved insertion modification of genomic sites by use of the double-guide editrons described herein. FIG. 3A shows schematic diagrams illustrating single-cut editrons 315 and 316 like those typically used for CRISPR modification of genomic sites compared to double-cut editron described herein (e.g., the 317 editron). FIG. 3B illustrates improved insertion by the double-cut 317 editron of a GFP coding region into the ADE2 genetic locus. The modified genomic fragments were detected by polymerase chain reaction (PCR) amplification of the ADE2 genomic locus after treatment with cas nuclease complexes that included the different 315, 316 and 317 gRNAs. The replaced fragments were smaller than the wild type or inserted fragments and traveled further through the polyacrylamide gel used for fragment separation (see diagram above the gel image). As illustrated, use of the double-cut 317 gRNAs provided significantly improved insertion frequency compared to the single cut 315 and 316 gRNAs.

### DETAILED DESCRIPTION

Described herein are modified retron nucleic acids that are useful for genomic editing. Modified retrons are useful sources of donor DNA for genomic editing because retron DNA can be made abundantly in vivo by reverse transcription from retron RNA using the retron's own reverse transcriptase or another reverse transcriptase expressed in the cell.

The modified retron nucleic acids are referred to as editing retrons (editrons). The editrons include a modified retron non-coding RNA (ncRNA) having a sequence for a first guide RNA, a sequence for a second guide RNA, a first RNA template for a first homology arm DNA, and a second RNA template for a second homology arm DNA. The first homology arm DNA and the second homology arm DNA are, for example, separately complementary to distinct sites of a target genomic site. The editing retron (editron) can further include an RNA template for a donor DNA that can be reverse transcribed by a reverse transcriptase. The donor DNA is complementary to a coding strand of a target gene, or complementary to the coding strand flanking regions. The first RNA template for a first homology arm DNA can flanks one end of a template for a donor DNA and the second RNA template for a second homology arm DNA can flank the second end of the template for the donor DNA. The RNA template for a donor DNA can also include an initiation site for a reverse transcriptase.

Also described herein are constructs, expression systems, and methods for precisely deleting, inserting and/or replacing genomic DNA within cells.

As illustrated herein, large insertions and/or deletions of genomic sites can efficiently be made using the editrons and methods described herein. Such large insertions and/or deletions can involve deletion/insertion of at least 25 nucleotides, at least 50 nucleotides, at least 75 nucleotides, at least 100 nucleotides, at least 125 nucleotides, at least 150 nucleotides, at least 175 nucleotides, at least 200 nucleotides, at least 250 nucleotides, at least 300 nucleotides, at least 350 nucleotides, at least 400 nucleotides, at least 500 nucleotides, at least 600 nucleotides, at least 700 nucleotides, at least 800 nucleotides, at least 900 nucleotides, at least 1000 nucleotides, at least 1500 nucleotides, at least 2000 nucleotides, at least 3000 nucleotides, at least 4000 nucleotides, at least 5000 nucleotides, at least 6000 nucleotides, at least 7000 nucleotides, at least 8000 nucleotides, at least 9000 nucleotides, at least 10,000 nucleotides, or more.

### Retrons

A retron is a distinct DNA found in the genomes of many bacteria species. A retron encodes a reverse transcriptase and a unique single-stranded DNA/RNA hybrid called multicopy single-stranded DNA (msDNA). The msDNA is an extrachromosomal satellite DNA that consists of a single-stranded DNA molecule covalently linked via a 2′-5′phosphodiester bond to an internal guanosine of an RNA molecule. Wild type retrons are about 2 kb long. They contain a single operon controlling the synthesis of an RNA transcript carrying three loci, msr, msd, and ret, that are involved in msDNA synthesis. The RNA of a retron DNA/RNA hybrid is referred to as a non-coding RNA (ncRNA, encoded by the msr). The ncRNA provides a template for synthesis of msDNA (msd) by the reverse transcriptase encoded the ret locus.

While msDNA and reverse transcribed DNA (RT-DNA) are related, the term reverse transcribed DNA (RT-DNA) is used herein to refer to any retron-related reverse transcribed DNA, whether modified or not, while the term msDNA refers to wild type, natural, or unmodified retron msDNA.

The ncRNA includes a pre-msr sequence, an msr gene encoding multicopy single-stranded RNA (msRNA). The msd gene encodes a multicopy single-stranded DNA (msDNA), the post-msd sequence, and a ret gene encoding a reverse transcriptase. Synthesis of DNA by the retron-encoded reverse transcriptase provides a DNA/RNA chimeric product which is composed of single-stranded DNA encoded by the msd gene linked to single-stranded RNA encoded by the msr gene. The retron msr RNA contains a conserved guanosine residue at the end of a stem loop structure. A strand of the msr RNA is joined to the 5′ end of the msd single-stranded DNA by a 2′-5′ phosphodiester linkage at the 2′ position of this conserved guanosine residue.

For example, a wild type retron-Eco1 ncRNA (also called ec86 or retron-Eco1 ncRNA) can have the sequence shown below as SEQ ID NO:1.

| | |
|---|---|
| 1 | TGCGCACCCT TAGCGAGAGG TTTATCATTA AGGTCAACCT |
| 41 | CTGGATGTTG TTTCGGCATC CTGCATTGAA TCTGAGTTAC |
| 81 | TGTCTGTTTT CCTTGTTGGA ACGGAGAGCA TCGCCTGATG |
| 121 | CTCTCCGAGC CAACCAGGAA ACCCGTTTTT TCTGACGTAA |
| 161 | GGGTGCGCA |

An example of an Eco1 human-codon optimized reverse transcriptase (RT) sequence that can be used is shown below as SEQ ID NO:2.

| | |
|---|---|
| 1 | ATGAAATCTG CAGAGTATCT GAATACGTTC CGCCTTAGGA |
| 41 | ATTTGGGCCT CCCCGTGATG AACAATCTCC ACGATATGAG |
| 81 | CAAGGCGACT CGAATATCCG TGGAAACGCT GAGACTGCTC |
| 121 | ATCTATACAG CAGACTTTCG GTACAGGATC TACACGGTCG |
| 161 | AAAAGAAGGG GCCTGAGAAA CGCATGCGAA CAATTTATCA |
| 201 | ACCTAGCCGA GAGCTCAAGG CGTTGCAGGG CTGGGTTCTT |
| 241 | CGAAACATCC TTGACAAACT CTCATCATCA CCCTTTAGTA |
| 281 | TTGGGTTTGA AAAGCACCAA AGCATCCTTA ACAACGCGAC |
| 321 | GCCACACATA GGTGCCAATT TCATATTGAA CATCGACTTG |
| 361 | GAGGATTTTT TTCCGAGCCT CACAGCCAAT AAAGTGTTCG |
| 401 | GTGTTTTTCA CAGTCTTGGG TACAATCGCC TTATTAGTTC |
| 411 | CGTTCTTACC AAGATTTGTT GTTACAAGAA TCTCTTGCCC |
| 481 | CAGGGAGCAC CCAGCAGTCC GAAATTGGCG AATTTGATTT |
| 521 | GTTCCAAGCT CGATTATCGA ATACAAGGGT ACGCGGGCAG |
| 561 | CCGGGGACTC ATCTATACCC GCTACGCAGA CGATCTTACG |
| 601 | CTGTCTGCCC AATCAATGAA GAAGGTCGTA AAGGCGCGGG |
| 641 | ATTTCTTGTT TTCTATCATC CCGTCCGAGG GCTTGGTAAT |
| 681 | TAATTCCAAA AAGACTTGTA TCTCAGGACC ACGATCTCAG |
| 721 | CGAAAAGTGA CAGGACTCGT CATTTCTCAA GAAAAAGTCG |
| 761 | GTATAGGGAG AGAGAAGTAT AAGGAAATCC GCGCGAAGAT |
| 801 | CCACCACATA TTCTGTGGCA AGAGCAGCGA GATAGAACAC |
| 841 | GTCCGAGGCT GGTTGTCCTT CATACTGAGC GTGGACTCAA |
| 881 | AAAGCCACCG CCGGTTGATC ACCTATATTT CAAAACTGGA |
| 921 | AAAGAAATAT GGAAAGAACC CACTCAACAA AGCTAAAACA |
| 961 | TAG |

An example of an Eco2 human-codon optimized reverse transcriptase (RT) sequence is shown below as SEQ ID NO:3.

| | |
|---|---|
| 1 | ATGACAAAAA CTTCAAAGCT GGATGCGCTG CGGGCGGCTA |
| 41 | CTAGTAGGGA AGATTTGGCG AAGATTCTCG ACATAAAGTT |
| 81 | GGTGTTTCTG ACAAACGTGT TGTACCGCAT AGGATCCGAC |
| 121 | AACCAGTATA CGCAATTCAC AATACCCAAA AAGGGTAAAG |
| 161 | GTGTCCGCAC CATCAGCGCA CCAACGGACC GACTTAAGGA |
| 201 | TATACAGAGG AGGATTTGTG ATCTTCTTAG TGACTGTAGG |
| 241 | GATGAAATCT TTGCGATTAG GAAGATCTCT AATAATTACT |
| 281 | CATTCGGCTT CGAAAGAGGA AAATCAATTA TACTCAATGC |
| 321 | TTACAAGCAT CGAGGGAAGC AAATTATATT GAACATCGAC |
| 361 | CTTAAGGACT TCTTTGAGAG CTTTAACTTT GGGAGAGTCC |
| 401 | GGGGGTACTT TCTCTCCAAC CAGGACTTCT TGTTGAACCC |
| 441 | AGTTGTGGCA ACAACGTTGG CGAAGGCCGC CTGCTACAAC |
| 481 | GGGACTCTGC CTCAGGGGTC CCCATGTTCC CCTATTATAA |
| 521 | GTAACCTTAT CTGTAACATT ATGGACATGC GGCTCGCAAA |
| 561 | GCTCGCCAAG AAGTACGGCT GCACTTATAG TCGATATGCG |
| 601 | GATGACATTA CGATCAGCAC CAATAAAAAT ACCTTCCCGT |
| 641 | TGGAGATGGC GACTGTGCAG CCTGAAGGGG TTGTGCTGGG |
| 681 | CAAAGTGCTC GTAAAGGAGA TTGAAAATTC AGGTTTCGAG |
| 721 | ATTAACGATT CTAAGACTAG ATTGACCTAC AAAACAAGTA |
| 761 | GGCAAGAAGT CACCGGGCTG ACGGTTAATC GGATTGTAAA |
| 801 | CATTGATCGG TGCTACTACA AAAAGACGAG GGCGCTGGCT |
| 841 | CACGCATTGT ATCGGACAGG AGAATATAAG GTCCCAGACG |
| 881 | AGAACGGTGT TCTGGTATCT GGAGGGCTTG ACAAGTTGGA |
| 921 | GGGTATGTTT GGGTTTATCG ACCAGGTGGA TAAATTCAAC |
| 961 | AACATTAAAA AAAAGTTGAA TAAGCAACCC GACAGATATG |
| 1001 | TTCTGACAAA TGCCACTTTG CACGGATTTA AGCTCAAATT |
| 1041 | GAACGCCAGG GAGAAAGCCT ATAGCAAATT CATCTACTAC |
| 1081 | AAATTCTTCC ACGGTAATAC TTGTCCCACG ATCATAACAG |
| 1121 | AGGGTAAGAC GGATAGGATT TACCTTAAAG CTGCCCTCCA |
| 1161 | TAGCCTCGAG ACAAGTTATC CTGAACTGTT TCGGGAGAAA |
| 1201 | ACAGATAGTA AGAAGAAGGA GATAAATCTG AATATTTTTA |
| 1241 | AAAGCAATGA GAAGACCAAG TATTTCCTGG ATCTCAGCGG |
| 1281 | CGGCACAGCA GACCTCAAGA AATTCGTGGA ACGCTACAAA |
| 1321 | AATAACTACG CTTCCTATTA CGGCAGCGTA CCGAAACAAC |
| 1361 | CGGTGATAAT GGTGCTTGAT AACGACACAG GCCCGTCAGA |
| 1401 | CCTGTTGAAC TTTTTGAGAA ACAAAGTTAA GAGTTGTCCA |
| 1441 | GATGATGTAA CAGAAATGCG CAAGATGAAG TACATACATG |
| 1481 | TGTTTTACAA TCTGTACATA GTTCTGACTC CCCTGTCTCC |
| 1521 | ATCTGGAGAG CAAACGTCTA TGGAGGACCT CTTTCCTAAA |
| 1561 | GATATATTGG ACATTAAGAT AGATGGCAAG AAATTCAATA |
| 1601 | AAAACAATGA CGGTGACTCC AAAACAGAGT ATGGGAAGCA |
| 1001 | CATATTCTCA ATGCGCGTTG TACGAGATAA AAAGAGGAAG |
| 1001 | ATAGATTTCA AGGCATTTTG CTGTATCTTC GATGCTATTA |
| 1001 | AGGATATTAA AGAACATTAC AAACTGATGT TGAATTCCTA |
| 1001 | G |

An example of an Eco1 wild type retron reverse transcriptase sequence is shown below as SEQ ID NO:4.

| | |
|---|---|
| 1 | KSAEYLNTFR LRNLGLPVMN NLHDMSKATR ISVETLRLLI |
| 41 | YTADFRYRIY TVEKKGPEKR MRTIYQPSRE LKALQGWVLR |
| 81 | NILDKLSSSP FSIGFEKHQS ILNNATPHIG ANFILNIDLE |
| 121 | DFFPSLTANK VFGVFHSLGY NRLISSVLTK ICCYKNLLPQ |
| 161 | GAPSSPKLAN LICSKLDYRI QGYAGSRGLI YTRYADDLTL |
| 201 | SAQSMKKVVK ARDFLFSIIP SEGLVINSKK TCISGPRSQR |
| 241 | KVTGLVISQE KVGIGREKYK EIRAKIHHIF CGKSSEIEHV |
| 281 | RGWLSFILSV DSKSHRRLIT YISKLEKKYG KNPLNKAKT |

An example of an Eco2 wild type retron reverse transcriptase sequence is shown below as SEQ ID NO:5.

| | |
|---|---|
| 1 | MTKTSKLDAL RAATSREDLA KILDIKLVFL TNVLYRIGSD |
| 41 | NQYTQFTIPK KGKGVRTISA PTDRLKDIQR RICDLLSDCR |
| 81 | DEIFAIRKIS NNYSFGFERG KSIILNAYKH RGKQIILNID |
| 121 | LKDFFESFNF GRVRGYFLSN QDFLLNPVVA TTLAKAACYN |
| 161 | GTLPQGSPCS PIISNLICNI MDMRLAKLAK KYGCTYSRYA |
| 201 | DDITISTNKN TFPLEMATVQ PEGVVLGKVL VKEIENSGFE |
| 241 | INDSKTRLTY KTSRQEVTGL TVNRIVNIDR CYYKKTRALA |
| 281 | HALYRTGEYK VPDENGVLVS GGLDKLEGMF GFIDQVDKFN |
| 321 | NIKKKLNKQP DRYVLTNATL HGFKLKLNAR EKAYSKFIYY |
| 361 | KFFHGNTCPT IITEGKTDRI YLKAALHSLE TSYPELFREK |
| 401 | TDSKKKEINL NIFKSNEKTK YFLDLSGGTA DLKKFVERYK |
| 441 | NNYASYYGSV PKQPVIMVLD NDTGPSDLLN FLRNKVKSCP |
| 481 | DDVTEMRKMK YIHVFYNLYI VLTPLSPSGE QTSMEDLFPK |
| 521 | DILDIKIDGK KFNKNNDGDS KTEYGKHIFS MRVVRDKKRK |
| 561 | IDFKAFCCIF DAIKDIKEHY KLMLNS |

An example of a sequence for an Eco4 retron reverse transcriptase is shown below as SEQ ID NO:6.

| | |
|---|---|
| 1 | MSIDIETTLQ KAYPDFDVLL KSRPATHYKV YKIPKRTIGY |
| 41 | RIIAQPTPRV KAIQRDIIEI LKQHTHIHDA ATAYVDGKNI |
| 81 | LDNAKIHQSS VYLLKLDLVN FFNKITPELL FKALARQKVD |
| 121 | ISDTNKNLLK QFCFWNRTKR KNGALVLSVG APSSPFISNI |
| 161 | VMSSFDEEIS SFCKENKISY SRYADDLTFS TNERDVLGLA |
| 201 | HQKVKTTLIR FFGTRIIINN NKIVYSSKAH NRHVTGVTLT |
| 241 | NNNKLSLGRE RKRYITSLVF KFKEGKLSNV DINHLRGLIG |
| 281 | FAYNIEPAFI ERLEKKYGES TIKSIKKYSE GG |

An example of a sequence for a Sen2 retron reverse transcriptase is shown below as SEQ ID NO:7.

| | |
|---|---|
| 1 | MDILQHISDL LLTKKSEIIS FSLTAPYRYK IYKIAKRNSD |
| 41 | KKRTIAHPSK ELKFIQREIT EYLTDKLPVH ECAFAYKKGS |
| 81 | SIKTNAQVHL HTKYLLKMDF ENFFPSITPR LFFSKLRLAN |
| 121 | IDLTADDKVL LENILFFKSK RNSNLRLSIG APSSPLISNF |
| 161 | VMYFWDIEVQ EICSKIGVNY TRYADDLTFS TNNKDVLFDI |
| 201 | PDMLENVLPK YSLGRIRINH EKTVFSSKGH NRHVTGITLT |
| 241 | NDNKLSIGRE RKRKISAMIH HFINGKLSTD ECNKLVGLLA |
| 281 | FAKNIEPSFY KSMVIKYGSD NIYKLQKQKD K |

Other types of retron types and retron components are described herein and can be used in the constructs, expression systems, and methods described herein.

In some cases, the modified ncRNA has the secondary structures that are substantially preserved relative to the unmodified retron ncRNA. In addition, the a1/a2 region are typically present in the modified ncRNAs, although such a1/a2 regions can be modified. For example, while RNA stem and loop features as well as the a1/a2 regions are present they can for example be lengthened. Such stem and loop features and such a1/a2 regions should not be entirely deleted or be so destabilized that the integrity of these secondary structures is lost. In other words, the secondary structures of the ncRNA should not be so destabilized that it becomes degraded either during in vitro preparation or in vivo.

Modified (e.g., engineered) ncRNAs can have alterations in different locations relative to the corresponding wild type ncRNAs. However, not every modification provides a stable ncRNA or one that can yield good amounts of reverse transcribed DNA.

One example of a location for modification of retron ncRNA is within a self-complementary region (stem region, which has sequence complementarity to the pre-msr sequence), wherein the length of the self-complementary region can be lengthened relative to the corresponding ncRNA of a native retron. Such modifications should retain the complementarity of the stem structure. The inventors have determined that lengthening retron stem regions results in an engineered retron that can provide enhanced production of RT-DNA.

In certain embodiments, the complementary stem region has a length at least 1, at least 2, at least 4, at least 6, at least 8, at least 10, at least 12, at least 14, at least 16, at least 18, at least 20, at least 30, at least 40, or at least 50 nucleotides longer than the wild-type self-complementary region. For example, the self-complementary region may have a length ranging from 1 to 50 nucleotides longer than the native or wild-type complementary region, including any length within this range, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 34, 35, 36, 37, 38, 39, 40, 41, 42, 43, 44, 45, 46, 47, 48, 49, 50, or more nucleotides longer. In certain embodiments, the self-complementary region has a length ranging from 1 to 16 nucleotides longer than the wild-type complementary region. The single-stranded DNA generated by the engineered retron ncRNA can be used in various applications.

To create more abundant RT-DNA, for example, the ncRNA SEQ ID NO:8 sequence shown below, with the native self-complementary 3′ and 5′ ends highlighted in bold (at positions 1-12 and 158-169), can be extended at positions 1 and 169 to extend the self-complementary region.

| | |
|---|---|
| 1 | TGCGCACCCT TAGCGAGAGG TTTATCATTA AGGTCAACCT |
| 41 | CTGGATGTTG TTTCGGCATC CTGCATTGAA TCTGAGTTAC |
| 81 | TGTCTGTTTT CCTTGTTGGA ACGGAGAGCA TCGCCTGATG |
| 121 | CTCTCCGAGC CAACCAGGAA ACCCGTTTTT TCTGACGTAA |
| 161 | GGGTGCGCA |

For example, as shown below for the following engineered “ncRNA extended” (SEQ ID NO:9), where the additional nucleotides that extend the self-complementary region are shown in italics with underlining.

| | |
|---|---|
| 1 | TGATAAGATT CCGTATGCGC ACCCTTAGCG AGAGGTTTAT |
| 41 | CATTAAGGTC AACCTCTGGA TGTTGTTTCG GCATCCTGCA |
| 81 | TTGAATCTGA GTTACTGTCT GTTTTCCTTG TTGGAACGGA |
| 121 | GAGCATCGCC TGATGCTCTC CGAGCCAACC AGGAAACCCG |
| 161 | TTTTTTCTGA CGTAAGGGTG CGCATACGGA ATCTTATCA |

In some cases, the additional nucleotides can be added to any position in the self-complementary region, for example, anywhere within positions 1-12 and 158-169 of the SEQ ID NO:8 or SEQ ID NO:9 sequence.

In certain embodiments, sequences of the ncRNA, msr gene, msd gene, and ret gene used in the engineered retron may be derived from any bacterial retron operon. Representative retrons are available such as those from gram-negative bacteria including, without limitation, myxobacteria retrons such as Myxococcus xanthus retrons (e.g., Mx65, Mx162) and Stigmatella aurantiaca retrons (e.g., Sa163); Escherichia coli retrons (e.g., Ec48, E67, Ec73, Ec78, EC83, EC86, EC107, and Ec107); Salmonella enterica; Vibrio cholerae retrons (e.g., Vc81, Vc95, Vc137); Vibrio parahaemolyticus (e.g., Vc96); and Nannocystis exedens retrons (e.g., Ne144). Retron ncRNA, msr gene, msd gene, and ret gene nucleic acid sequences as well as retron reverse transcriptase protein sequences may be derived from any source. Representative retron sequences, including ncRNA, msr gene, msd gene, and ret gene nucleic acid sequences and reverse transcriptase protein sequences are listed in the National Center for Biotechnology Information (NCBI) database. See, for example, NCBI entries: Accession Nos. EF428983, M55249, EU250030, X60206, X62583, AB299445, AB436696, AB436695, M86352, M30609, M24392, AF427793, AQ3354, and AB079134; all of which sequences (as entered by the date of filing of this application) are herein incorporated by reference in their entireties.

The retron ncRNAs can be modified to enhance production of retron reverse transcribed DNA in a host cell or to provide host cells with genomic editing components or other useful proteins and/or nucleic acids. Any of the foregoing retron sequences (or variants thereof) can include variant or mutant nucleotides, added nucleotides, or fewer nucleotides.

For example, a parental ncRNA can be modified by addition of nucleotides to a stem or loop as described herein. Before modification the parental ncRNA can have at least about 80-100% sequence identity to any region of the retrons described herein, including any percent identity within this range, such as 81, 82, 83, 84, 85, 86, 87, 88, 89, 90, 91, 92, 93, 94, 95, 96, 97, 98, or 99% sequence identity to any region of the retron sequences described herein (including those defined by accession number). Such parental retrons can be used to construct an engineered retron or vector system comprising an engineered retron, as described herein.

The variant ncRNAs can include exogenous or heterologous nucleotides or nucleic acid segments. For example, the exogenous or heterologous nucleotide or nucleic acid segments can add at least 1, at least 2, at least 4, at least 6, at least 8, at least 10, at least 12, at least 14, at least 16, at least 18, at least 20, at least 30, at least 40, at least 50, at least 60, at least 70, at least 80, at least 90, at least 100, at least 125, at least 150, at least 175, or at least 200 nucleotides to parental retron nucleic acids, to thereby generate variant retron nucleic acids.

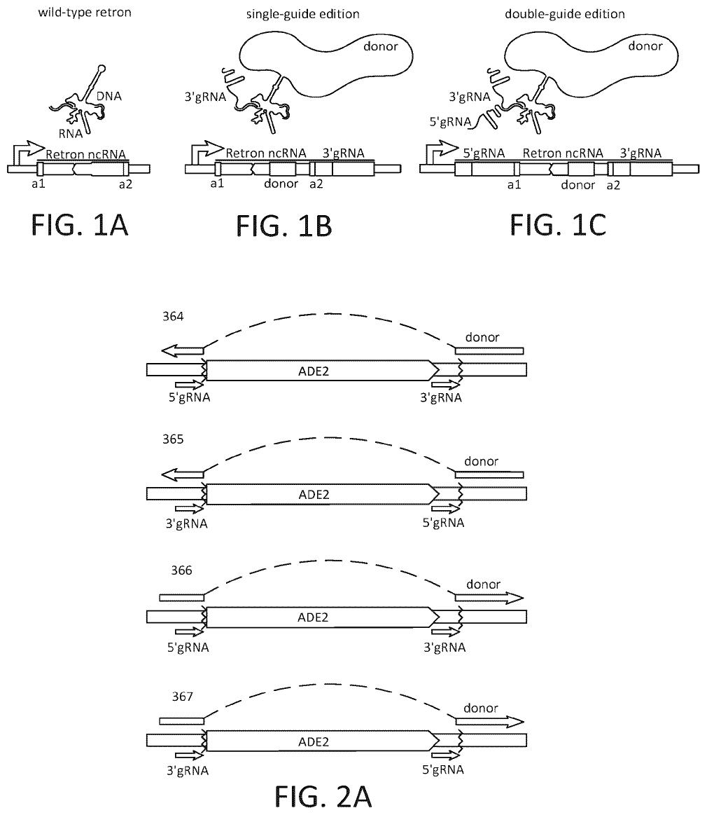

As described herein, the retron nucleic acids can be modified with respect to the native retron to include one or more heterologous sequences of interest, including two guide RNAs and a donor polynucleotide suitable for use in gene editing, e.g., by homology directed repair (HDR) or recombination-mediated genetic engineering (recombineering). The two guide RNAs (e.g., with the tracrRNA) can, for example, be separately linked to the 5′ and 3′ ends of the retron nucleic acids. In some cases, the donor DNA sequence of interest can be inserted into the loop of the msd stem loop of the retron or a loop of the ncRNA (see, e.g., FIG. 1C).

Such heterologous sequences (guide RNA templates, donor DNA templates) may be inserted, for example, into the ncRNA coding region of an expression cassette. Upon transcription, the ncRNA will contain the guide RNAs, as well as the RNA segment encoding the donor DNA. The ncRNA can be partially reverse transcribed to generate the donor DNA.

### Retron Expression Systems

Modified retrons, retron nucleic acids, ncRNAs, or retron constructs can be incorporated into and expressed from an expression cassette or expression vector. In general, the selected retron nucleic acids include or encode one or more wild type or modified ncRNA, retron reverse transcriptases, as well as libraries or populations thereof. The retrons or retron libraries can be expressed from expression cassettes or expression vectors that can be present in vitro or in vivo within host cells.

Modified retron ncRNAs, msr genes, msd genes, and/or ret genes can individually or collectively be expressed in vivo from an expression cassette or expression vector within a cell. As described herein the modified ncRNAs can include a sequence for a first guide RNA, a sequence for a second guide RNA, a first RNA template for a first homology arm DNA, and a second RNA template for a second homology arm DNA. The modified ncRNAs can also include a sequence for a donor DNA (that can be reverse transcribed by a reverse transcriptase).

A “vector” is a composition of matter that can be used to deliver a nucleic acid of interest to the interior of a cell. Retron (modified and/or unmodified) nucleic acids can be introduced into a cell with a single vector or in multiple separate vectors to produce wild type, mutant or modified retron RNA (ncRNA) and/or DNA and/or reverse transcriptases in host cells. Vectors typically include control elements operably linked to the retron sequences, which allow for expression in vivo in the host cells. For example, the segment encoding the modified retron ncRNA and/or the segment encoding the ret (reverse transcriptase) can be operably linked to the same or different promoters to allow expression of the modified retron ncRNA, and/or the retron reverse transcriptase. The retron donor DNA can be reverse transcribed from the ncRNA to provide a reverse transcribed DNA (RT-DNA).

In some embodiments, heterologous sequences encoding desired products of interest (e.g., guide RNAs, donor polynucleotides for gene editing, and combinations thereof) may be inserted in the segment encoding the ncRNA.

Any eukaryotic, archeon, or prokaryotic cell, capable of being transfected with a vector comprising the engineered retron sequences, may be used as host cells for the retron-related expression cassettes and expression vectors. The ability of constructs to express ncRNA, donor DNA, or other retron-encoded products (e.g., reverse transcriptases) can be empirically determined using the methods described herein.

In some embodiments, the modified retron nucleic acids are produced by a vector system comprising one or more vectors. In the vector system, the modified ncRNA and the reverse transcriptase may be provided by the same vector (i.e., cis arrangement of such retron elements), wherein the vector comprises a promoter operably linked to the segment encoding the ncRNA and the segment encoding the reverse transcriptase. In some embodiments, a second promoter is operably linked to the segment encoding the reverse transcriptase. Alternatively, the segment encoding the reverse transcriptase may be incorporated into a second vector that does not include the ncRNA, msr gene or the msd gene (i.e., trans arrangement).

Numerous vectors are available including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated virus vectors, retroviral vectors, lentiviral vectors, and the like. An expression construct can be replicated in a living cell, or it can be made synthetically. For purposes of this application, the terms “expression construct,” “expression vector,” and “vector,” are used interchangeably to demonstrate the application of the invention in a general, illustrative sense, and are not intended to limit the invention.

In certain embodiments, the nucleic acid comprising one or more wild type or modified retron sequences is under transcriptional control of a promoter. A “promoter” refers to a DNA sequence recognized by the synthetic machinery of the cell, or introduced synthetic machinery, required to initiate the specific transcription of a gene. The term promoter will be used here to refer to a group of transcriptional control modules that are clustered around the initiation site for RNA polymerase I, II, or III. Typical promoters for mammalian cell expression include the SV40 early promoter, a CMV promoter such as the CMV immediate early promoter (see, U.S. Pat. Nos. 5,168,062 and 5,385,839, incorporated herein by reference in their entireties), the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression. These and other promoters can be obtained from commercially available plasmids, using techniques well known in the art. See, e.g., Sambrook et al., supra. Enhancer elements may be used in association with the promoter to increase expression levels of the constructs. Examples include the SV40 early gene enhancer, as described in Dijkema et al., EMBO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al., Cell (1985) 41:521, such as elements included in the CMV intron A sequence.

Expression vectors for expressing one or more retron nucleic acids can include a promoter “operably linked” to a nucleic acid segment encoding the ncRNA and/or the reverse transcriptase. The phrase “operably linked” or “under transcriptional control” as used herein means that the promoter is in the correct location and orientation in relation to a polynucleotide to control the initiation of transcription by RNA polymerase and expression of the ncRNA and/or the reverse transcriptase.

Typically, transcription terminator/polyadenylation signals will also be present in the expression construct. Examples of such sequences include, but are not limited to, those derived from SV40, as described in Sambrook et al., supra, as well as a bovine growth hormone terminator sequence (see, e.g., U.S. Pat. No. 5,122,458). Additionally, 5′-UTR sequences can be placed adjacent to the coding sequence in order to enhance expression of the same. Such sequences may include UTRs comprising an internal ribosome entry site (IRES).

Inclusion of an IRES permits the translation of one or more open reading frames from a vector. Such an IRES element attracts a eukaryotic ribosomal translation initiation complex and promotes translation initiation. See, e.g., Kaufman et al., Nuc. Acids Res. (1991) 19:4485-4490; Gurtu et al., Biochem. Biophys. Res. Comm. (1996) 229:295-298; Rees et al., BioTechniques (1996) 20:102-110; Kobayashi et al., BioTechniques (1996) 21:399-402; and Mosser et al., BioTechniques (1997 22: 150-161. A multitude of IRES sequences are available and include sequences derived from a wide variety of viruses, such as from leader sequences of picornaviruses such as the encephalomyocarditis virus (EMCV) UTR (Jang et al. J. Virol. (1989) 63:1651-1660), the polio leader sequence, the hepatitis A virus leader, the hepatitis C virus IRES, human rhinovirus type 2 IRES (Dobrikova et al., Proc. Natl. Acad. Sci. (2003) 100(25):15125-15130), an IRES element from the foot and mouth disease virus (Ramesh et al., Nucl. Acid Res. (1996) 24:2697-2700), a giardiavirus IRES (Garlapati et al., J. Biol. Chem. (2004) 279(5):3389-3397), and the like. A variety of nonviral IRES sequences will also find use herein, including, but not limited to IRES sequences from yeast, as well as the human angiotensin II type 1 receptor IRES (Martin et al., Mol. Cell Endocrinol. (2003) 212:51-61), fibroblast growth factor IRESs (FGF-1 IRES and FGF-2 IRES, Martineau et al. (2004) Mol. Cell. Biol. 24(17):7622-7635), vascular endothelial growth factor IRES (Baranick et al. (2008) Proc. Natl. Acad. Sci. U.S.A. 105(12):4733-4738, Stein et al. (1998) Mol. Cell. Biol. 18(6):3112-3119, Bert et al. (2006) RNA 12(6):1074-1083), and insulin-like growth factor 2 IRES (Pedersen et al. (2002) Biochem. J. 363(Pt 1):37-44). These elements are readily commercially available in plasmids sold, e.g., by Clontech (Mountain View, CA), Invivogen (San Diego, CA), Addgene (Cambridge, MA) and GeneCopoeia (Rockville, MD). See also IRESite: The database of experimentally verified IRES structures (iresite.org). An IRES sequence may be included in a vector, for example, to express a reverse transcriptase or an RNA-guided nuclease (e.g., Cas9) from an expression cassette.

Alternatively, a polynucleotide encoding a viral 2A-self cleaving peptide can be used to allow production of multiple protein products (e.g., Cas9, retron reverse transcriptase, etc.) from a single vector. One or more 2A linker peptides can be inserted between the coding sequences in the multicistronic construct. The 2A peptide, which is self-cleaving, allows co-expressed proteins from the multicistronic construct to be produced at equimolar levels. 2A peptides from various viruses may be used, including, but not limited to 2A peptides derived from the foot-and-mouth disease virus, equine rhinitis A virus, Thosea asigna virus and porcine teschovirus-1. See, e.g., Kim et al. (2011) PLoS One 6(4):e18556, Trichas et al. (2008) BMC Biol. 6:40, Provost et al. (2007) Genesis 45(10):625-629, Furler et al. (2001) Gene Ther. 8(11):864-873; herein incorporated by reference in their entireties.

In certain embodiments, the expression construct comprises a plasmid sequence suitable for transforming a bacterial host. Numerous bacterial expression vectors are available. Bacterial expression vectors include, but are not limited to, pACYC177, pASK75, pBAD, pBADM, pBAT, pCal, pET, pETM, pGAT, pGEX, pHAT, pKK223, pMal, pProEx, pQE, and pZA31. Bacterial plasmids may contain antibiotic selection markers (e.g., ampicillin, kanamycin, erythromycin, carbenicillin, streptomycin, or tetracycline resistance), a lacZ gene (β-galactosidase produces blue pigment from x-gal substrate), fluorescent markers (e.g., GFP. mCherry), or other markers for selection of transformed bacteria. See, e.g., Sambrook et al., supra.

In other embodiments, the expression construct comprises a plasmid suitable for transforming a yeast cell. Yeast expression plasmids typically contain a yeast-specific origin of replication (ORI) and nutritional selection markers (e.g., HIS3, URA3, LYS2, LEU2, TRP1, MET15, ura4+, leu1+, ade6+), antibiotic selection markers (e.g., kanamycin resistance), fluorescent markers (e.g., mCherry), or other markers for selection of transformed yeast cells. The yeast plasmid may further contain components to allow shuttling between a bacterial host (e.g., E. coli) and yeast cells. A number of different types of yeast plasmids are available including yeast integrating plasmids (YIp), which lack an ORI and are integrated into host chromosomes by homologous recombination; yeast replicating plasmids (YRp), which contain an autonomously replicating sequence (ARS) and can replicate independently; yeast centromere plasmids (YCp), which are low copy vectors containing a part of an ARS and part of a centromere sequence (CEN); and yeast episomal plasmids (YEp), which are high copy number plasmids comprising a fragment from a 2 micron circle (a natural yeast plasmid) that allows for 50 or more copies to be stably propagated per cell.

In other embodiments, the expression construct comprises a virus or engineered construct derived from a viral genome. A number of viral based systems have been developed for gene transfer into mammalian cells. These include adenoviruses, retroviruses (γ-retroviruses and lentiviruses), poxviruses, adeno-associated viruses, baculoviruses, and herpes simplex viruses (see e.g., Warnock et al. (2011) Methods Mol. Biol. 737:1-25; Walther et al. (2000) Drugs 60(2):249-271; and Lundstrom (2003) Trends Biotechnol. 21(3):117-122; herein incorporated by reference in their entireties). The ability of certain viruses to enter cells via receptor-mediated endocytosis, to integrate into host cell genomes and express viral genes stably and efficiently have made them attractive candidates for the transfer of foreign genes into mammalian cells.

For example, retroviruses provide a convenient platform for gene delivery systems. Selected sequences can be inserted into a vector and packaged in retroviral particles. The recombinant virus can then be isolated and delivered to host cells, or cells of a selected subject either in vivo or ex vivo. A number of retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; Boris-Lawrie and Temin (1993) Cur. Opin. Genet. Develop. 3:102-109; and Ferry et al. (2011) Curr. Pharm. Des. 17(24):2516-2527). Lentiviruses are a class of retroviruses that are particularly useful for delivering polynucleotides to mammalian cells because they are able to infect both dividing and nondividing cells (see e.g., Lois et al (2002) Science 295:868-872; Durand et al. (2011) Viruses 3(2):132-159; herein incorporated by reference).

A number of adenovirus vectors can be used. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476). Additionally, various adeno-associated virus (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor Laboratory Press); Carter, B. J. Current Opinion in Biotechnology (1992) 3:533-539; Muzyczka, N. Current Topics in Microbiol. and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875.

Another vector system useful for delivering nucleic acids encoding the modified retron nucleic acids and/or reverse transcriptases is the enterically administered recombinant poxvirus vaccines described by Small, Jr., P. A., et al. (U.S. Pat. No. 5,676,950, issued Oct. 14, 1997, herein incorporated by reference).

Additional viral vectors which will find use for delivering the nucleic acid molecules of interest include those derived from the pox family of viruses, including vaccinia virus and avian poxvirus. By way of example, vaccinia virus recombinants expressing a nucleic acid molecule of interest (e.g., engineered retron) can be constructed as follows. The DNA encoding the particular nucleic acid sequence is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the sequences of interest into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can also be used to deliver the nucleic acid molecules of interest. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the alphavirus genus, such as, but not limited to, vectors derived from the Sindbis virus (SIN), Semliki Forest virus (SFV), and Venezuelan Equine Encephalitis virus (VEE), will also find use as viral vectors for delivering the polynucleotides of the present invention. For a description of Sindbis-virus derived vectors useful for the practice of the instant methods, see, Dubensky et al. (1996) J. Virol. 70:508-519; and International Publication Nos. WO 95/07995, WO 96/17072; as well as Dubensky, Jr., T. W., et al., U.S. Pat. No. 5,843,723, issued Dec. 1, 1998, and Dubensky, Jr., T. W., U.S. Pat. No. 5,789,245, issued Aug. 4, 1998, both herein incorporated by reference. Particularly preferred are chimeric alphavirus vectors comprised of sequences derived from Sindbis virus and Venezuelan equine encephalitis virus. See, e.g., Perri et al. (2003) J. Virol. 77: 10394-10403 and International Publication Nos. WO 02/099035, WO 02/080982, WO 01/81609, and WO 00/61772; herein incorporated by reference in their entireties.

A vaccinia-based infection/transfection system can be conveniently used to provide for inducible, transient expression of the nucleic acids of interest (e.g., engineered retron) in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the nucleic acid of interest, driven by a T7 promoter. The polymerase expressed in the cytoplasm from the vaccinia virus recombinant transcribes the transfected DNA into RNA. The method provides for high level, transient, cytoplasmic production of large quantities of RNA. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

As an alternative approach to infection with vaccinia or avipox virus recombinants, or to the delivery of nucleic acids using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more templates. Concomitantly, there can be modified retron nucleic acids whose expression is under the control of the T7 promoter. Thus, some of the T7 RNA polymerase generated from translation of the amplification template RNA will lead to transcription of the desired retron ncRNAs and/or retron reverse transcriptases. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

Insect cell expression systems, such as baculovirus systems, can also be used and are known to those of skill in the art and described in, e.g., Baculovirus and Insect Cell Expression Protocols (Methods in Molecular Biology, D. W. Murhammer ed., Humana Press, 2^{nd} edition, 2007) and L. King, The Baculovirus Expression System: A laboratory guide (Springer, 1992). Materials and methods for baculovirus/insect cell expression systems are commercially available in kit form from, inter alia, Thermo Fisher Scientific (Waltham, MA) and Clontech (Mountain View, CA).

Plant expression systems can also be used for transforming plant host cells. Generally, such systems use virus-based vectors to transfect plant cells with heterologous genes. For a description of such systems see, e.g., Porta et al., Mol. Biotech. (1996) 5:209-221; and Hackland et al., Arch. Virol. (1994) 139:1-22.

In order to effect expression of engineered retron constructs, the expression construct can be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsulated in an infectious viral particle.

Several non-viral methods for the transfer of expression constructs into cultured cells also are contemplated. These include the use of calcium phosphate precipitation, DEAE-dextran, electroporation, direct microinjection, DNA-loaded liposomes, lipofectamine-DNA complexes, cell sonication, gene bombardment using high velocity microprojectiles, and receptor-mediated transfection (see, e.g., Graham and Van Der Eb (1973) Virology 52:456-467; Chen and Okayama (1987) Mol. Cell Biol. 7:2745-2752; Rippe et al. (1990) Mol. Cell Biol. 10:689-695; Gopal (1985) Mol. Cell Biol. 5:1188-1190; Tur-Kaspa et al. (1986) Mol. Cell. Biol. 6:716-718; Potter et al. (1984) Proc. Natl. Acad. Sci. USA 81:7161-7165); Harland and Weintraub (1985) J. Cell Biol. 101:1094-1099); Nicolau & Sene (1982) Biochim. Biophys. Acta 721:185-190; Fraley et al. (1979) Proc. Natl. Acad. Sci. USA 76:3348-3352; Fechheimer et al. (1987) Proc Natl. Acad. Sci. USA 84:8463-8467; Yang et al. (1990) Proc. Natl. Acad. Sci. USA 87:9568-9572; Wu and Wu (1987) J. Biol. Chem. 262:4429-4432; Wu and Wu (1988) Biochemistry 27:887-892; herein incorporated by reference). Some of these techniques may be successfully adapted for in vivo or ex vivo use.

Delivery of retron nucleic acids to a cell can generally be accomplished with or without vectors. The retrons, retron nucleic acids, or vectors containing them may be introduced into any type of cell, including any cell from a prokaryotic, eukaryotic, or archaeon organism, including bacteria, archaea, fungi, protists, plants (e.g., monocotyledonous and dicotyledonous plants), and animals (e.g., vertebrates and invertebrates). Examples of animal cells that may be transfected with an engineered retron include, without limitation, cells from vertebrates such as fish, birds, mammals (e.g., human and non-human primates, farm animals, pets, and laboratory animals), reptiles, and amphibians. Examples of plant cells that may be transfected with an engineered retron include, without limitation, cells from crops including cereals such as wheat, oats, and rice, legumes such as soybeans and peas, corn, grasses such as alfalfa, and cotton. The engineered retrons can be introduced into a single cell or a population of cells of interest. Cells from tissues, organs, and biopsies, as well as recombinant cells, genetically modified cells, cells from cell lines cultured in vitro, and artificial cells (e.g., nanoparticles, liposomes, polymersomes, or microcapsules encapsulating nucleic acids) may all be transfected with the engineered retrons. The subject methods are also applicable to cellular fragments, cell components, or organelles (e.g., mitochondria in animal and plant cells, plastids (e.g., chloroplasts) in plant cells and algae). Cells may be cultured or expanded after transfection with the engineered retron constructs.

A variety of methods for introducing nucleic acids into a host cell are available. Commonly used methods include chemically induced transformation, typically using divalent cations (e.g., CaCl₂)), dextran-mediated transfection, polybrene mediated transfection, lipofectamine and LT-1 mediated transfection, electroporation, protoplast fusion, encapsulation of nucleic acids in liposomes, and direct microinjection of the nucleic acids comprising engineered retrons into nuclei. See, e.g., Sambrook et al. (2001) Molecular Cloning, a laboratory manual, 3^{rd} edition, Cold Spring Harbor Laboratories, New York, Davis et al. (1995) Basic Methods in Molecular Biology, 2^{nd} edition, McGraw-Hill, and Chu et al. (1981) Gene 13:197; herein incorporated by reference in their entireties.

Once the expression construct has been delivered into the cell the vector or cassette comprising the retron nucleic acids may be positioned and expressed at different sites. In certain embodiments, the vector or cassette comprising the retron nucleic acids may be stably integrated into the genome of the cell. This integration may be in the cognate location and orientation, or it may be integrated in a random, non-specific location (gene augmentation). In yet further embodiments, the vector or cassette comprising the retron nucleic acids may be stably maintained in the cell as a separate, episomal segment of DNA. Such nucleic acid segments or “episomes” encode sequences sufficient to permit maintenance and replication independent of or in synchronization with the host cell cycle. How the vector or cassette comprising the retron nucleic acids are delivered to a cell and where in the cell the nucleic acid remains is dependent on the type of expression construct employed.

In yet another embodiment, the expression construct may simply consist of naked recombinant DNA or plasmids comprising the retron nucleic acids (e.g., expression cassettes). Transfer of the constructs may be performed by any of the methods mentioned above which physically or chemically permeabilize the cell membrane. This is particularly applicable for transfer in vitro but it may be applied to in vivo use as well. Dubensky et al. (Proc. Natl. Acad. Sci. USA (1984) 81:7529-7533) successfully injected polyomavirus DNA in the form of calcium phosphate precipitates into liver and spleen of adult and newborn mice demonstrating active viral replication and acute infection. Benvenisty & Neshif (Proc. Natl. Acad. Sci. USA (1986) 83:9551-9555) also demonstrated that direct intraperitoneal injection of calcium phosphate-precipitated plasmids results in expression of the transfected genes. It is envisioned that DNA encoding retron nucleic acids of interest may also be transferred in a similar manner in vivo and express retron products.

In other cases, a naked DNA expression construct may be transferred into cells by particle bombardment. This method depends on the ability to accelerate DNA-coated microprojectiles to a high velocity allowing them to pierce cell membranes and enter cells without killing them (Klein et al. (1987) Nature 327:70-73). Several devices for accelerating small particles have been developed. One such device relies on a high voltage discharge to generate an electrical current, which in turn provides the motive force (Yang et al. (1990) Proc. Natl. Acad. Sci. USA 87:9568-9572). The microprojectiles may consist of biologically inert substances, such as tungsten or gold beads.

In a further embodiment, the expression construct may be delivered using liposomes. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh & Bachhawat (1991) Liver Diseases, Targeted Diagnosis and Therapy Using Specific Receptors and Ligands, Wu et al. (Eds.), Marcel Dekker, NY, 87-104). Also contemplated is the use of lipofectamine-DNA complexes.

In certain embodiments, the liposome may be complexed with a hemagglutinin virus (HVJ). This has been shown to facilitate fusion with the cell membrane and promote cell entry of liposome-encapsulated DNA (Kaneda et al. (1989) Science 243:375-378). In other embodiments, the liposome may be complexed or employed in conjunction with nuclear non-histone chromosomal proteins (HMG-I) (Kato et al. (1991) J. Biol. Chem. 266(6):3361-3364). In yet further embodiments, the liposome may be complexed or employed in conjunction with both HVJ and HMG-I. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu (1993) Adv. Drug Delivery Rev. 12:159-167).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin (see, e.g., Wu and Wu (1987), supra; Wagner et al. (1990) Proc. Natl. Acad. Sci. USA 87(9):3410-3414). A synthetic neoglycoprotein, which recognizes the same receptor as ASOR, has been used as a gene delivery vehicle (Ferkol et al. (1993) FASEB J. 7:1081-1091; Perales et al. (1994) Proc. Natl. Acad. Sci. USA 91(9):4086-4090), and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (Methods Enzymol. (1987) 149:157-176) employed lactosyl-ceramide, a galactose-terminal asialoganglioside, incorporated into liposomes and observed an increase in the uptake of the insulin gene by hepatocytes. Thus, it is feasible that a nucleic acid encoding a particular gene also may be specifically delivered into a cell by any number of receptor-ligand systems with or without liposomes. Also, antibodies to surface antigens on cells can similarly be used as targeting moieties.

In a particular example, a recombinant polynucleotide comprising retron nucleic acids may be administered in combination with a cationic lipid. Examples of cationic lipids include, but are not limited to, lipofectin, DOTMA, DOPE, and DOTAP. The publication of WO/0071096, which is specifically incorporated by reference, describes different formulations, such as a DOTAP:cholesterol or cholesterol derivative formulation that can effectively be used for gene therapy. Other disclosures also discuss different lipid or liposomal formulations including nanoparticles and methods of administration; these include, but are not limited to, U.S. Patent Publication 20030203865, 20020150626, 20030032615, and 20040048787, which are specifically incorporated by reference to the extent they disclose formulations and other related aspects of administration and delivery of nucleic acids. Methods used for forming particles are also disclosed in U.S. Pat. Nos. 5,844,107, 5,877,302, 6,008,336, 6,077,835, 5,972,901, 6,200,801, and 5,972,900, which are incorporated by reference for those aspects.

### Genomic Editing

The methods described herein can perform genomic editing by using clustered regularly interspaced short palindromic repeats (CRISPR)/CRISPR-associated (Cas) systems. CRISPR/Cas systems are useful, for example, for RNA-programmable genome editing (see e.g., Marraffini and Sontheimer. Nature Reviews Genetics 11: 181-190 (2010); Sorek et al. Nature Reviews Microbiology 2008 6: 181-6; Karginov and Hannon. Mol Cell 2010 1:7-19; Hale et al. Mol Cell 2010:45:292-302; Jinek et al. Science 2012 337:815-820; Bikard and Marraffini Curr Opin Immunol 2012 24:15-20; Bikard et al. Cell Host & Microbe 2012 12: 177-186; all of which are incorporated by reference herein in their entireties).

Homology arms and donor DNA facilitate genomic editing of a target genomic site. As demonstrated herein the orientation of the guide RNAs and donor DNA relative to the genomic locus can affect the efficiency of genomic editing. Upon synthesis the homology arms and donor DNA should be complementary to one target strand. As illustrated herein genomic editing is significantly reduced when the guide RNAs do not bind the target strand replaced by the donor DNA/homology donor DNA. For example, approximately 2-fold to 100-fold improved deletion of a genomic sites can be achieved when the reverse transcribed donor DNA was produced in the reverse direction relative to the orientation of the guide RNAs, i.e., when the guide RNAs bind the target strand that is replaced by the donor DNA/homology arms. In some cases, the dual cutting, optimally oriented editrons described herein provide at least 2-fold, at least 3-fold, at least 4-fold, at least 5-fold, at least 7-fold, at least 10-fold, at least 15-fold, at least 20-fold, at least 25-fold, at least 30-fold, at least 40-fold, at least 50-fold, at least 60-fold, at least 70-fold, at least 80-fold, at least 90-fold, at least 100-fold, or at least 200-fold improved genomic deletions and/or insertions are achieved compared to single cutter guide RNA methods.

A CRISPR guide RNA system is adapted for use with the constructs, modified retron nucleic acids, and the methods described herein. The guide RNAs can include two components: a CRISPR RNA (crRNA), which is a 17-20 nucleotide sequence complementary to the target DNA, and a trans-activating crRNA (tracrRNA) that is a binding scaffold for the Cas nuclease. In some cases, the two components are fused to make a single guide RNA (sgRNA). The tracrRNA forms a stem loop that is recognized and bound by the cas nuclease. The crRNA typically has shorter sequence than the tracrRNA. The term “guide RNA” as used herein refers to either a single guide RNA (sgRNA, with both crRNA and tracrRNA components) or a crRNA. The CRISPR technique is generally described, for example, by Mali et al. Science 339:823-6 (2013); which is incorporated by reference herein in its entirety.

The two guide RNAs are encoded within or adjacent to the ncRNA coding region of the expression cassettes, for example, one guide RNA at the 5′ end of the ncRNA and the other guide RNA at the 3′ end of the ncRNA.

Upon transcription of the guide RNAs, each guide RNA can target a Cas enzyme to the desired location in the genome, where it can cleave the genomic DNA for generation of a genomic modification. Donor DNA encoded within the retron ncRNA and reverse transcribed within the host cells modifies (e.g., repairs) the genomic target site.

There are several types of CRISPR systems, some of which are summarized in the chart below.

CRISPR System Types Overview

| System | Features | Examples |
|---|---|---|
| Type I | Multiple proteins (5-7 proteins | Staphylococcus epidermidis |
| | typical), crRNA. DNA Cleavage | (Type IA) |
| | is catalyzed by Cas3. | |
| Type II | 3-4 proteins (one protein (Cas9) | Streptococcus pyogenes |
| | has nuclease activity) two RNAs. | CRISPR/Cas9, Francisella |
| | Target DNA cleavage catalyzed by | novicida U112 Cpf1 |
| | Cas9 and RNA components. | |
| Type III | Five or six proteins required for | S. epidermidis (Type IIIA); |
| | cutting, number of required RNAs | P. furiosus; (Type IIIB). |
| | unknown but expected to be one. | |
| | Type IIIB systems have the ability | |
| | to target RNA. | |

In some cases, the cas nuclease is a Type II CRISPR endonuclease. The term “Class II CRISPR endonuclease” refers to endonucleases that have similar endonuclease activity as Cas9 and participate in a Class II CRISPR system. The Cas9 nuclease can, for example, be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonfex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

An example of a Class II CRISPR system is the type II CRISPR locus from Streptococcus pyogenes SF370, which contains a cluster of four genes Cas9, Cas1, Cas2, and Csn1, as well as a tracrRNA and a characteristic array of repetitive sequences (direct repeats) interspaced by short stretches of non-repetitive sequences (spacers, about 30 bp each). In this system, targeted DNA double-strand break (DSB) may be generated in four sequential steps. First, the pre-crRNA array and tracrRNA, may be transcribed from the expression cassette that encodes the ncRNA and the guide RNA. Second, tracrRNA may hybridize to the direct repeats of pre-CRISPR guide RNA (pre-crRNA), which is then processed into mature crRNAs containing individual spacer sequences. Third, the mature crRNA:tracrRNA complex can direct Cas9 to the DNA target consisting of the protospacer and the corresponding PAM sequence via heteroduplex formation between the spacer region of the crRNA and the protospacer DNA. Finally, Cas9 may mediate cleavage of target DNA upstream of PAM to create a double-stranded break within the protospacer.

A “guide RNA” or “gRNA” as provided herein refers to a ribonucleotide sequence capable of binding a cas nuclease, thereby forming ribonucleoprotein complex. The gRNA includes a nucleotide sequence complementary to a target site (e.g., near or at a genomic site to be edited). In some cases, the guide RNA includes one or more RNA molecules. TracrRNAs can be used to facilitate assembly of a ribonucleoprotein complex that includes the gRNA together with the tracrRNA and a cas nuclease. A complementary nucleotide sequence of the guide RNA can mediate binding of the ribonucleoprotein complex to the target site thereby providing the sequence specificity of the ribonucleoprotein complex. Thus, the guide RNA includes a sequence that is complementary to a target nucleic acid sequence such that the guide RNA binds a target nucleic acid sequence.

In some cases, the complement of the guide RNA includes a sequence having a sequence identity of about 50%, 55%, 60%, 65%, 70%, 75%, 80%, 85%, 90%, 95%, 96%, 97%, 98% or 99% to a target nucleic acid (e.g., a target genomic DNA sequence). In some cases, a target nucleic acid sequence is a nucleic acid sequence expressed by a cell. In some cases, the target nucleic acid sequence is an exogenous nucleic acid sequence. In some cases, the target nucleic acid sequence is an endogenous nucleic acid sequence. In some cases, the target nucleic acid sequence forms part of a cellular gene. In some cases, the target nucleic acid sequence is a genomic DNA site or location. Thus, some cases, the guide RNA is complementary to a cellular gene or fragment thereof. In some cases, the guide RNA includes a sequence having sequence identity of about 50%, 55%, 60%, 65%, 70%, 75%, 80%, 85%, 90%, 95%, 96%, 97%, 98% or 99% to the target nucleic acid sequence. In some cases, the guide RNA includes a sequence that is about 50%, 55%, 60%, 65%, 70%, 75%, 80%, 85%, 90%, 95%, 96%, 97%, 98% or 99% complementary to the sequence of a cellular gene. In some cases, the guide RNA binds a cellular gene target sequence. In some cases, the guide RNA or complement thereof, includes a sequence having a sequence identity of at least about 90%, 95%, or 100% to a target nucleic acid.

In some cases, segment bound by a guide RNA within the target nucleic acid is about or at least about 10, 15, 20, 25, or more nucleotides in length.

The guide RNA is a single-stranded ribonucleic acid, although in some cases it may form some double-stranded regions by folding onto itself. In some cases, the guide RNA is about 10, 20, 30, 40, 50, 60, 70, 80, 90, 100 or more nucleic acid residues in length. In some cases, the guide RNA is from about 10 to about 30 nucleic acid residues in length. In some cases, the guide RNA is about 20 nucleic acid residues in length. For example, the length of the guide RNA can be at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides or residues in length. In some cases, the guide RNA is from 5 to 50, 10 to 50, 15 to 50, 20 to 50, 25 to 50, 30 to 50, 35 to 50, 40 to 50, 45 to 50, 5 to 75, 10 to 75, 15 to 75, 20 to 75, 25 to 75, 30 to 75, 35 to 75, 40 to 75, 45 to 75, 50 to 75, 55 to 75, 60 to 75, 65 to 75, 70 to 75, 5 to 100, 10 to 100, 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, 95 to 100, or more nucleotides or residues in length. In some cases, the guide RNA is from 10 to 15, 10 to 20, 10 to 30, 10 to 40, or 10 to 50 residues in length.

### Definitions

The term “about” as used herein when referring to a measurable value such as an amount, a length, and the like, is meant to encompass variations of 20% or +10%, more preferably +5%, even more preferably +1%, and still more preferably +0.1% from the specified value.

“Recombinant” as used herein to describe a nucleic acid molecule means a polynucleotide of retron, genomic, cDNA, bacterial, semisynthetic, or synthetic origin which, by virtue of its origin or manipulation, is not associated with all or a portion of the polynucleotide with which it is associated in nature.

The term “recombinant” as used with respect to a protein or polypeptide means a polypeptide produced by expression of a recombinant polynucleotide. In general, the polynucleotide of interest is cloned and then expressed in transformed organisms, for example, as described herein. The host organism expresses the foreign nucleic acids to produce the RNA, RT-DNA, or protein under expression conditions.

As used herein, a “cell” refers to any type of cell isolated from a prokaryotic, eukaryotic, or archaeon organism, including bacteria, archaea, fungi, protists, plants, and animals, including cells from tissues, organs, and biopsies, as well as recombinant cells, cells from cell lines cultured in vitro, and cellular fragments, cell components, or organelles comprising nucleic acids. The term also encompasses artificial cells, such as nanoparticles, liposomes, polymersomes, or microcapsules encapsulating nucleic acids. The methods described herein can be performed, for example, on a sample comprising a single cell or a population of cells. The term also includes genetically modified cells.

The term “transformation” refers to the insertion of an exogenous polynucleotide (e.g., an engineered retron) into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Recombinant host cells,” “host cells”, “cells”, “cell lines”, “cell cultures”, and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence which “encodes” a selected polypeptide or a selected RNA, is a nucleic acid molecule which is transcribed (in the case of DNA templates) into RNA and/or translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, ncRNAs, tracrRNAs, ncRNAs modified to include heterologous sequences, cDNA from viral, prokaryotic or eukaryotic ncRNA, mRNA, genomic DNA sequences from retron, viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper polymerases are present. The promoter need not be contiguous with the coding sequence, so long as it functions to direct the expression thereof. Thus, for example, intervening untranslated yet transcribed sequences can be present between the promoter sequence and the coding sequence, and the promoter sequence can still be considered “operably linked” to the coding sequence.

“Encoded by” refers to a nucleic acid sequence which codes for a polypeptide or RNA sequence. For example, the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. The RNA sequence or a portion thereof contains a nucleotide sequence of at least 3 to 5 nucleotides, more preferably at least 8 to 10 nucleotides, and even more preferably at least 15 to 20 nucleotides.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein, DNA, or RNA or cause other adverse consequences. That is, a nucleic acid or peptide of this invention is purified if it is substantially free of cellular material, viral material, or culture medium when obtained from nature or when produced by recombinant DNA techniques, or free from chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

“Substantially purified” generally refers to isolation of a substance (nucleic acid, compound, polynucleotide, protein, polypeptide, peptide composition) such that the substance comprises the majority percent of the sample in which it resides. Typically, in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

“Purified polynucleotide” or “purified nucleic acid” refers to a polynucleotide or nucleic acid of interest or fragment thereof which is essentially free, e.g., contains less than about 50%, preferably less than about 70%, and more preferably less than about at least 90%, of the protein and/or nucleic acids with which the polynucleotide is naturally associated. Techniques for purifying polynucleotides of interest are available in the art and include, for example, disruption of the cell containing the polynucleotide with a chaotropic agent and separation of the polynucleotide(s) and proteins by ion-exchange chromatography, affinity chromatography and sedimentation according to density.

The term “transfection” is used to refer to the uptake of foreign DNA by a cell. A cell has been “transfected” when exogenous DNA has been introduced inside the cell membrane. A number of transfection techniques are generally available. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material and includes uptake of peptide-linked or antibody-linked DNAs.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

“Expression” refers to detectable production of a gene product by a cell. The gene product may be a transcription product (i.e., RNA), which may be referred to as “gene expression”, or the gene product may be a translation product of the transcription product (i.e., a protein), depending on the context.

“Mammalian cell” refers to any cell derived from a mammalian subject suitable for transfection with retron nucleic acids or vector systems comprising retron nucleic acids, as described herein. The cell may be xenogeneic, autologous, or allogeneic. The cell can be a primary cell obtained directly from a mammalian subject. The cell may also be a cell derived from the culture and expansion of a cell obtained from a mammalian subject. Immortalized cells are also included within this definition. In some embodiments, the cell has been genetically engineered to express a recombinant protein and/or nucleic acid.

The term “subject” includes animals, including both vertebrates and invertebrates, including, without limitation, invertebrates such as arthropods, mollusks, annelids, and cnidarians; and vertebrates such as amphibians, including frogs, salamanders, and caecillians; reptiles, including lizards, snakes, turtles, crocodiles, and alligators; fish; mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, geese, and the like. In some cases, the disclosed methods find use in experimental animals, in veterinary application, and in the development of animal models for disease, including, but not limited to, rodents including mice, rats, and hamsters; primates, and transgenic animals.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses.

The term “derived from” is used herein to identify the original source of a molecule but is not meant to limit the method by which the molecule is made which can be, for example, by chemical synthesis or recombinant means.

A polynucleotide or nucleic acid “derived from” a designated sequence refers to a polynucleotide or nucleic acid that includes a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

The terms “hybridize” and “hybridization” refer to the formation of complexes between nucleotide sequences which are sufficiently complementary to form complexes via Watson-Crick base pairing.

The term “homologous region” refers to a region of a nucleic acid with homology to another nucleic acid region. Thus, whether a “homologous region” is present in a nucleic acid molecule is determined with reference to another nucleic acid region in the same or a different molecule. Further, since a nucleic acid is often double stranded, the term “homologous, region,” as used herein, refers to the ability of nucleic acid molecules to hybridize to each other. For example, a single-stranded nucleic acid molecule can have two homologous regions which are capable of hybridizing to each other. Thus, the term “homologous region” includes nucleic acid segments with complementary sequences. Homologous regions may vary in length but will typically be between 4 and 500 nucleotides (e.g., from about 4 to about 40, from about 40 to about 80, from about 80 to about 120, from about 120 to about 160, from about 160 to about 200, from about 200 to about 240, from about 240 to about 280, from about 280 to about 320, from about 320 to about 360, from about 360 to about 400, from about 400 to about 440, etc.).

As used herein, the terms “complementary” or “complementarity” refers to polynucleotides that are able to form base pairs with one another. Base pairs are typically formed by hydrogen bonds between nucleotide units in an anti-parallel orientation between polynucleotide strands. Complementary polynucleotide strands can base pair in a Watson-Crick manner (e.g., A to T, A to U, C to G), or in any other manner that allows for the formation of duplexes. As persons skilled in the art are aware, when using RNA as opposed to DNA, uracil (U) rather than thymine (T) is the base that is considered to be complementary to adenosine. However, when uracil is denoted in the context of the present invention, the ability to substitute a thymine is implied, unless otherwise stated. “Complementarity” may exist between two RNA strands, two DNA strands, or between an RNA strand and a DNA strand. It is generally understood that two or more polynucleotides may be “complementary” and able to form a duplex despite having less than perfect or less than 100% complementarity. Two sequences are “perfectly complementary” or “100% complementary” if at least a contiguous portion of each polynucleotide sequence, comprising a region of complementarity, perfectly base pairs with the other polynucleotide without any mismatches or interruptions within such region. Two or more sequences are considered “perfectly complementary” or “100% complementary” even if either or both polynucleotides contain additional non-complementary sequences as long as the contiguous region of complementarity within each polynucleotide is able to perfectly hybridize with the other. “Less than perfect” complementarity refers to situations where less than all of the contiguous nucleotides within such region of complementarity are able to base pair with each other. Determining the percentage of complementarity between two polynucleotide sequences is a matter of ordinary skill in the art.

The term “Cas9” as used herein encompasses type II clustered regularly interspaced short palindromic repeats (CRISPR) system Cas9 endonucleases from any species, and also includes biologically active fragments, variants, analogs, and derivatives thereof that retain Cas9 endonuclease activity (i.e., catalyze site-directed cleavage of DNA to generate double-strand breaks). A Cas9 endonuclease binds to and cleaves DNA at a site comprising a sequence complementary to its bound guide RNA (gRNA). For purposes of Cas9 targeting, a gRNA may comprise a sequence “complementary” to a target sequence (e.g., major or minor allele), capable of sufficient base-pairing to form a duplex (i.e., the gRNA hybridizes with the target sequence). Additionally, the gRNA may comprise a sequence complementary to a PAM sequence, wherein the gRNA also hybridizes with the PAM sequence in a target DNA.

The term “donor polynucleotide” or “donor DNA” refers to a nucleic acid or polynucleotide that provides a nucleotide sequence of an intended edit to be integrated into the genome at a target locus by HDR or recombineering.

A “target site” or “target sequence” is the nucleic acid sequence recognized (i.e., sufficiently complementary for hybridization) by a guide RNA (gRNA) or a homology arm of a donor polynucleotide (donor DNA). The target site may be allele-specific (e.g., a major or minor allele). For example, a target site can be a genomic site that is intended to be modified such as by insertion of one or more nucleotides, replacement of one or more nucleotides, deletion of one or more nucleotides, or a combination thereof. As illustrated herein, large insertions and/or deletions of genomic sites can be made using the editrons and methods described herein. Such large insertions and/or deletions can involve deletion/insertion of at least 25 nucleotides, at least 50 nucleotides, at least 75 nucleotides, at least 100 nucleotides, at least 125 nucleotides, at least 150 nucleotides, at least 175 nucleotides, at least 200 nucleotides, at least 250 nucleotides, at least 300 nucleotides, at least 350 nucleotides, at least 400 nucleotides, at least 500 nucleotides, at least 600 nucleotides, at least 700 nucleotides, at least 800 nucleotides, at least 900 nucleotides, at least 1000 nucleotides, at least 1500 nucleotides, at least 2000 nucleotides, at least 3000 nucleotides, at least 4000 nucleotides, at least 5000 nucleotides, at least 6000 nucleotides, at least 7000 nucleotides, at least 8000 nucleotides, at least 9000 nucleotides, at least 10,000 nucleotides, or more.

By “homology arm” is meant a portion of a donor polynucleotide that can facilitate targeting of the donor polynucleotide to the genomic sequence to be edited in a cell. The donor polynucleotide typically comprises a 5′ homology arm that hybridizes to a 5′ genomic target sequence and a 3′ homology arm that hybridizes to a 3′ genomic target sequence flanking a nucleotide sequence comprising the intended edit to the genomic DNA. The homology arms are referred to herein as 5′ and 3′ (i.e., upstream and downstream) homology arms, which relates to the relative position of the homology arms to the nucleotide sequence comprising the intended edit within the donor polynucleotide. The 5′ and 3′ homology arms hybridize to regions within the target locus in the genomic DNA to be modified, which are referred to herein as the “5′ target sequence” and “3′ target sequence,” respectively. For example, the nucleotide sequence comprising the intended edit can be integrated into the genomic DNA by HDR or recombineering at the genomic target locus recognized (i.e., sufficiently complementary for hybridization) by the 5′ and 3′ homology arms.

In general, “a CRISPR system” refers collectively to transcripts and other elements involved in the expression of or directing the activity of CRISPR-associated (“Cas”) genes, including sequences encoding a Cas gene. In some embodiments, one or more elements of a CRISPR system are derived from a type I, type II, or type III CRISPR system. Cas1 and Cas2 are found in all three types of CRISPR-Cas systems, and they are involved in spacer acquisition. In the I-E system of E. coli, Cas1 and Cas2 form a complex where a Cas2 dimer bridges two Cas1 dimers. In this complex Cas2 performs a non-enzymatic scaffolding role, binding double-stranded fragments of invading DNA, while Cas1 binds the single-stranded flanks of the DNA and catalyzes their integration into CRISPR arrays.

In some embodiments, one or more elements of a CRISPR system are derived from a particular organism comprising an endogenous CRISPR system, such as Streptococcus pyogenes. In general, a CRISPR system can be characterized by elements that promote the formation of a CRISPR complex at the site of a target sequence (also referred to as a protospacer in the context of an endogenous CRISPR system).

In some embodiments, a vector comprises a regulatory element operably linked to an enzyme-coding sequence encoding a CRISPR enzyme, such as a Cas protein. Non-limiting examples of Cas proteins include Casl, CaslB, Cas2, Cas3, Cas4, Cas5, Cas6, Cas7, Cas8, Cas9 (also known as Csnl and Csxl2), CaslO, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csxl7, Csxl4, CsxlO, Csxl6, CsaX, Csx3, Csxl, Csxl5, Csfl, Csf2, Csf3, Csf4, homologs thereof, or modified versions thereof.

In certain embodiments, the disclosure provides protospacers that are adjacent to short (3-5 bp) DNA sequences termed protospacer adjacent motifs (PAM). The PAMs are important for type I and type II systems during acquisition. In type I and type II systems, protospacers are excised at positions adjacent to a PAM sequence, with the other end of the spacer is cut using a ruler mechanism, thus maintaining the regularity of the spacer size in the CRISPR array. The conservation of the PAM sequence differs between CRISPR-Cas systems and may be evolutionarily linked to Cas1 and the leader sequence.

In one embodiment, the protospacer is a defined synthetic DNA. In some embodiments, the defined synthetic DNA is at least 3, 5, 10, 20, 30, 40, or 50 nucleotides, or between 3-50, or between 10-100, or between 20-90, or between 30-80, or between 40-70, or between 50-60, nucleotides in length. In one embodiment, the oligo nucleotide sequence or the defined synthetic DNA includes a modified “AAG” protospacer adjacent motif (PAM).

In some embodiments, a regulatory element is operably linked to one or more elements of a CRISPR system so as to drive expression of the one or more elements of the CRISPR system. In general, CRISPRs (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino et al, J. BacterioL, 169:5429-5433 (1987); and Nakata et al., J. BacterioL, 171:3553-3556 (1989)), and associated genes. Similar interspersed SSRs have been identified in Haloferax mediterranei, Streptococcus pyogenes, Anabaena, and Mycobacterium tuberculosis (See, Groenen et al., Mol. Microbiol., 10:1057-1065 (1993); Hoe et al., Emerg. Infect. Dis., 5:254-263 (1999); Masepohl et al, Biochim. Biophys. Acta 1307:26-30 (1996); and Mojica et al, Mol. Microbiol, 17:85-93 (1995)). The CRISPR loci typically differ from other SSRs by the structure of the repeats, which have been termed short regularly spaced repeats (SRSRs) (Janssen et al, OMICS J. Integ. Biol., 6:23-33 (2002); and Mojica et al, Mol. Microbiol., 36:244-246 (2000)). In general, the repeats are short elements that occur in clusters that are regularly spaced by unique intervening sequences with a substantially constant length (Mojica et al., (2000), supra). Although the repeat sequences are highly conserved between strains, the number of interspersed repeats and the sequences of the spacer regions typically differ from strain to strain (van Embden et al., J. Bacteriol., 182:2393-2401 (2000)). CRISPR loci have been identified in more than 40 prokaryotes (See e.g., Jansen et al, Mol. Microbiol., 43:1565-1575 (2002); and Mojica et al, (2005)) including, but not limited to Aeropyrum, Pyrobaculum, Sulfolobus, Archaeoglobus, Halocarcula, Methanobacteriumn, Methanococcus, Methanosarcina, Methanopyrus, Pyrococcus, Picrophilus, Thernioplasnia, Corynebacterium, Mycobacterium, Streptomyces, Aquifrx, Porphvromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myrococcus, Campylobacter, Wolinella, Acinetobacter, Erwinia, Escherichia, Legionella, Methylococcus, Pasteurella, Photobacterium, Salmonella, Xanthomonas, Yersinia, Treponema, and Thermotoga.

In some embodiments, an enzyme coding sequence encoding a CRISPR enzyme (e.g., cas9) is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about one or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding a CRISPR enzyme correspond to the most frequently used codon for a particular amino acid.

“Administering” a nucleic acid, such as an expression cassette, engineered retron construct or vector comprising an expression cassette or engineered retron construct to a cell comprises transducing, transfecting, electroporating, translocating, fusing, phagocytosing, shooting or ballistic methods, etc., i.e., any means by which a nucleic acid can be transported across a cell membrane.

The subject matter disclosed herein is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosed subject matter. The upper and lower limits of these smaller ranges may independently be included in the smaller ranges, and are also encompassed within the disclosed subject matter, subject to any specifically excluded limit in the stated range. Where the stated range includes one or both of the limits, ranges excluding either or both of those included limits are also included in the disclosed subject matter.

Unless defined otherwise, all technical and scientific terms used herein have the same meaning as commonly understood by one of ordinary skill in the art to which the disclosed subject matter belongs. Although any methods and materials similar or equivalent to those described herein can also be used in the practice or testing of the disclosed subject matter, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cell” includes a plurality of such cells and reference to “the nucleic acid” includes reference to one or more nucleic acids and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of any features or elements described herein, which includes use of a “negative” limitation.

It is appreciated that certain features of the disclosed subject matter, which are, for clarity, described in the context of separate embodiments, may also be provided in combination in a single embodiment. Conversely, various features of the disclosed subject matter, which are, for brevity, described in the context of a single embodiment, may also be provided separately or in any suitable sub-combination. All combinations of the embodiments pertaining to the disclosure are specifically embraced by the disclosed subject matter and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The following Examples illustrate some of the materials, methods, and experiments that were used or performed in the development of the invention.

### Example 1: Editrons can Efficiently Produce Deletions in Targeted Genomic Sites

This Example illustrates precise excision of a genomic locus flanked by two guide RNA targeted sites to generate a large deletion in a yeast chromosome. FIG. 1B-1C shows schematic diagrams illustrating the structures of ‘editron’ retrons modified to supply guide RNAs and donor DNAs for genomic editing. The expression cassettes shown at the bottom of each of FIG. 1B-1C illustrate expression of non-coding RNAs (ncRNAs) for the different editrons compared to a wild type retron ncRNA (FIG. 1A). As illustrated, a single-guide editron (FIG. 1B) can provide a single guide RNA for genomic editing, while a double-guide editron (FIG. 1C) can provide two guide RNAs. Each of the single-guide and double-guide editrons can encode a donor DNA (RT-DNA) that is reverse transcribed from the ncRNAs transcribed from the expression cassettes shown below each of FIG. 1B and FIG. 1C.

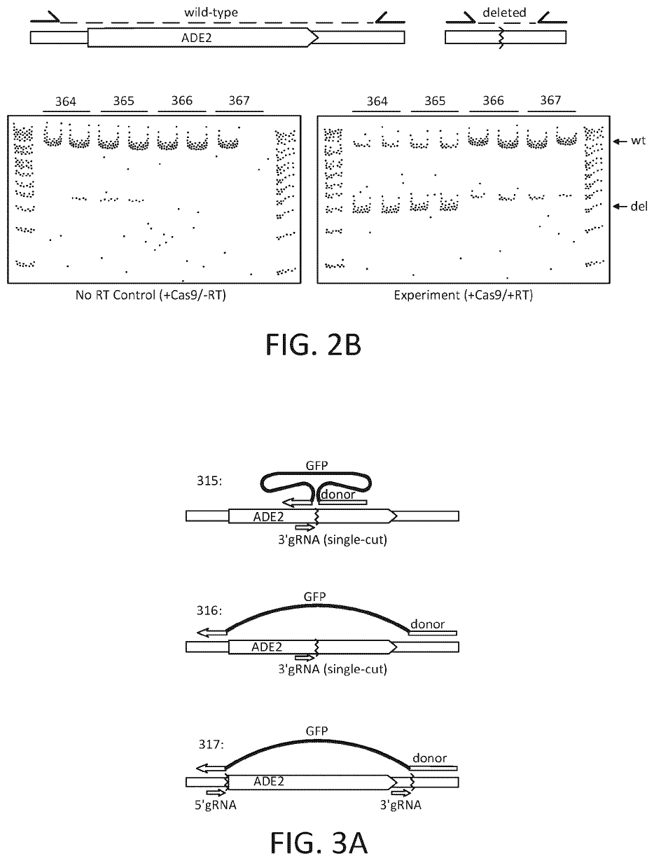

The guide RNAs of the editrons include sequences that are complementary to the site of genomic modification. For example, in some experiments the editrons were designed to have homology arms that extend away from the cutting sites of the two guide RNAs, as shown in FIG. 2A. These homology arms are sometimes referred to a donor DNA, even though they may not contribute significant DNA to the edited genomic site and may only facilitate deletion of the targeted genomic state.

Note that for constructs 364 and 365 the reverse transcribed donor (orange) was produced in the reverse direction relative to the orientation of the ADE2 gene and the gRNAs, whereas in constructs 366 and 367 the reverse transcribed donor is produced in the same orientation as the ADE2 gene and gRNAs. In other words, the guide RNAs for constructs 364 and 365 were designed to bind the target strand that is initially replaced by the single-stranded donor DNA, whereas the guide RNAs for constructs 366 and 367 were not.

The orientation of the reverse-transcribed donor DNA relative to the guide RNAs can in some cases be important because as shown in FIG. 2B, constructs 364 and 365 provided substantially improved genomic deletion relative to constructs 366 and 367. At least approximately 2-fold to 100-fold improved deletion of a genomic site therefore occurred when the reverse transcribed donor DNA was produced in the reverse direction relative to the orientation of the guide RNAs.

The constructs (editrons) described herein are therefore useful for precise and efficient excision of the targeted genomic loci.

### Example 2: Editrons can Efficiently Produce Insertions in Targeted Genomic Sites

As illustrated in FIG. 1B-1C, editrons can provide donor DNAs for insertion into a target genomic locus.

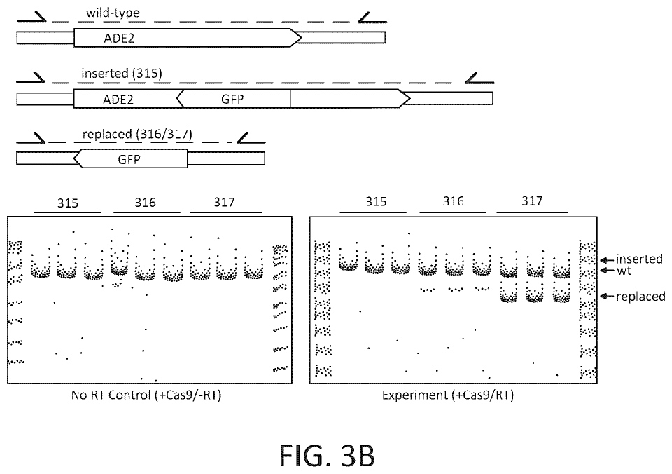

To evaluate the efficiency of genomic insertions by editrons, constructs 315, 316, and 317 were generated all having a template for an donor insertion DNA that encoded green fluorescent protein (GFP, FIG. 3A) designed to insert a GFP coding region into the yeast ADE2 gene. The single cutting retron editors were expressed from expression vectors pSCL315 and pSCL316, and they encode the same guide RNA(s) and donor DNAs but differ in the sequence of the RT-DNA encoded homology arms. The pSCL315 construct encodes homology arms that flank the guide RNA-defined cut site, while pSCL316 encodes homology arms flanking the ADE2 gene. In contrast, the pSCL317 expression vector was designed to encode a dual cutting retron editor, designed to both delete the ADE2 gene and insert a GFP gene in its place. Thus, the pSCL317 and pSCL316 expression cassettes encode the same homology arms, which flank the ADE2 gene, but differ in their guide RNA cut sites. The pSCL316 construct had a single guide RNA that induces DNA cleavage within the ADE2 gene, while pSCL317 had two guide RNAs that target regions flanking the ADE2 gene, inducing dual cuts in either side of the ADE2 gene. The CRISPR SpCas9 nuclease was expressed with a reverse transcriptase and either the single or dual-cutter ncRNAs. Therefore, as illustrated in FIG. 3A, constructs 315 and 316 provided only a single guide RNA targeted to the middle of the ADE2 gene, while construct 317 provided two guide RNAs, one targeted to the 5′ region and the other targeted to the 3′ region of the ADE2 gene. Moreover, construct 315 had homology arms targeted to bind sites abutting the target cleavage site, while construct 316 had homology arms that could bind the ADE gene 5′ and 3′ flanking regions. Construct 317 had homology arms that extended away from the cleavage sites of its two guide RNAs.

The genomic DNA targeting by guide RNAs and subsequent DNA repair was through endogenous repair machinery using the respective retron-derived DNA repair templates. The products of genomic editing were evaluated by PCR amplification using primers that flanked the ADE2 gene. The primers used either amplified the whole ADE2 gene, or a new genomic region, generated by either (1) the insertion of a GFP coding sequence into the ADE2 gene; or (2) the deletion of the ADE2 gene and the insertion of a GFP gene (smaller amplicon).

As shown in FIG. 3B, the 317 construct that provided two guide RNAs and homology arms flanking the guide RNA target sites efficiently replaced the ADE2 coding region with the GFP coding region. While low levels of GFP insertions were detected when the 315 construct was used, little or no genomic replacements were detected when the 315 or 316 constructs were used.

Accordingly, the dual guide RNA/complementary donor DNA provided by the editrons described herein are significantly more effective and efficient for generating genomic replacements than are single guide RNA homologous DNA methods that are currently in use for genomic editing.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following Statements summarize aspects and features of the invention.

### Statements:

1. An editing retron (editron) comprising a modified retron non-coding RNA (ncRNA) comprising a sequence for a first guide RNA, a sequence for a second guide RNA, a first RNA template for a first homology arm DNA, and a second RNA template for a second homology arm DNA.

2. The editing retron (editron) of statement 1, wherein the first homology arm DNA and the second homology arm DNA are separately complementary to distinct sites of a target genomic site.

3. The editing retron (editron) of statement 1 or 2, further comprising an RNA template for a donor DNA that can be reverse transcribed by a reverse transcriptase.

4. The editing retron (editron) of statement 3, wherein the reverse transcribed donor DNA is single stranded.

5. The editing retron (editron) of any one of statements 1-4, wherein the first RNA template for a first homology arm DNA flanks one end of a template for a donor DNA and the second RNA template for a second homology arm DNA flanks the second end of the template for the donor DNA.

6. The editing retron (editron) of any one of statements 3-5, wherein the first guide RNA and the second guide RNA bind to a target strand that is replaced by the reverse transcribed donor DNA.

7. The editing retron (editron) of any one of statements 3-6, wherein RNA template for a donor DNA comprises an initiation site for a reverse transcriptase.

8. An expression system comprising at least one expression cassette comprising a promoter operably linked to coding region for an editing retron (editron) comprising a modified retron non-coding RNA (ncRNA) comprising a sequence for a first guide RNA, a sequence for a second guide RNA, a first RNA template for a first homology arm DNA, and a second RNA template for a second homology arm DNA.

9. The expression system of statement 8, further comprising at least one expression cassette comprising a promoter operably linked to a coding region for a reverse transcriptase.

10. The expression system of statement 8 or 9, further comprising at least one expression cassette comprising a promoter operably linked to a coding region for a cas nuclease.

11. The expression system of any one of statements 8-10, wherein the first homology arm DNA and the second homology arm DNA are separately complementary to distinct sites of a target genomic site.

12. The expression system of any one of statements 8-11, wherein the editing retron (editron) further comprises an RNA template for a donor DNA that can be reverse transcribed by a reverse transcriptase.

13. The expression system of statement 12, wherein the reverse transcribed donor DNA is single stranded.

14. The expression system of any one of statements 8-13, wherein the first RNA template for a first homology arm DNA flanks one end of a template for a donor DNA and the second RNA template for a second homology arm DNA flanks the second end of the template for the donor DNA.

15. The expression system of any one of statements 12-14, wherein the first guide RNA and the second guide RNA bind to a target strand that is replaced by the reverse transcribed donor DNA.

16. The expression system of any one of statements 12-15, wherein the RNA template for a donor DNA comprises an initiation site for a reverse transcriptase.

17. A method comprising: (a) transforming a population of host cells, each host cell comprising a reverse transcriptase and a cas nuclease, with an expression system comprising at least one expression cassette comprising a promoter operably linked to coding region for an editing retron (editron) comprising a modified retron non-coding RNA (ncRNA) comprising a sequence for a first guide RNA, a sequence for a second guide RNA, a first RNA template for a first homology arm DNA, and a second RNA template for a second homology arm DNA.

18. The method of statement 17, wherein the first homology arm DNA and the second homology arm DNA are separately complementary to distinct sites of a target genomic site.

19. The method of statement 17 or 18, wherein the editing retron (editron) further comprises an RNA template for a donor DNA that can be reverse transcribed by a reverse transcriptase.

20. The method of statement 19, wherein the reverse transcribed donor DNA is single stranded.

21. The method of any one of statements 17-20, wherein the first RNA template for a first homology arm DNA flanks one end of a template for a donor DNA and the second RNA template for a second homology arm DNA flanks the second end of the template for the donor DNA.

22. The method of any one of statements 19-21, wherein the first guide RNA and the second guide RNA bind to a target strand that is replaced by the reverse transcribed donor DNA.

23. The method of any one of statements 19-22, wherein the RNA template for a donor DNA comprises an initiation site for a reverse transcriptase.

24. The method of any one of statements 17-23, which provides at least 2-fold, at least 3-fold, at least 4-fold, at least 5-fold, at least 7-fold, at least 10-fold, at least 15-fold, at least 20-fold, at least 25-fold, at least 30-fold, at least 40-fold, at least 50-fold, at least 60-fold, at least 70-fold, at least 80-fold, at least 90-fold, at least 100-fold, or at least 200-fold more genomic deletions, genomic replacements, and/or genomic insertions compared to use of a single cutter guide RNA.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

1. An editing retron (editron) comprising a modified retron non-coding RNA (ncRNA) comprising a sequence for a first guide RNA, a sequence for a second guide RNA, a first RNA template for a first homology arm DNA, and a second RNA template for a second homology arm DNA.

2. The editing retron (editron) of claim 1, wherein the first homology arm DNA and the second homology arm DNA are separately complementary to distinct sites of a target genomic site.

3. The editing retron (editron) of claim 1, further comprising an RNA template for a donor DNA that can be reverse transcribed by a reverse transcriptase.

4. The editing retron (editron) of claim 3, wherein the reverse transcribed donor DNA is single stranded.

5. The editing retron (editron) of claim 1, wherein the first RNA template for a first homology arm DNA flanks one end of a template for a donor DNA and the second RNA template for a second homology arm DNA flanks the second end of the template for the donor DNA.

6. The editing retron (editron) of claim 1, wherein the first guide RNA and the second guide RNA bind to a target strand that is replaced by the reverse transcribed donor DNA.

7. The editing retron (editron) of claim 3, wherein RNA template for a donor DNA comprises an initiation site for a reverse transcriptase.

8. An expression system comprising at least one expression cassette comprising a promoter operably linked to coding region for an editing retron (editron) comprising a modified retron non-coding RNA (ncRNA) comprising a sequence for a first guide RNA, a sequence for a second guide RNA, a first RNA template for a first homology arm DNA, and a second RNA template for a second homology arm DNA.

9. The expression system of claim 8, further comprising at least one expression cassette comprising a promoter operably linked to a coding region for a reverse transcriptase.

10. The expression system of claim 8, further comprising at least one expression cassette comprising a promoter operably linked to a coding region for a cas nuclease.

11. The expression system of claim 8, wherein the first homology arm DNA and the second homology arm DNA are separately complementary to distinct sites of a target genomic site.

12. The expression system of claim 8, wherein the editing retron (editron) further comprises an RNA template for a donor DNA that can be reverse transcribed by a reverse transcriptase.

13. The expression system of claim 12, wherein the reverse transcribed donor DNA is single stranded.

14. The expression system of claim 8, wherein the first RNA template for a first homology arm DNA flanks one end of a template for a donor DNA and the second RNA template for a second homology arm DNA flanks the second end of the template for the donor DNA.

15. The expression system of claim 12, wherein the first guide RNA and the second guide RNA bind to a target strand that is replaced by the reverse transcribed donor DNA.

16. The expression system of claim 12, wherein the RNA template for a donor DNA comprises an initiation site for a reverse transcriptase.

17. A method comprising: (a) transforming a population of host cells, each host cell comprising a reverse transcriptase and a cas nuclease, with an expression system comprising at least one expression cassette comprising a promoter operably linked to coding region for an editing retron (editron) comprising a modified retron non-coding RNA (ncRNA) comprising a sequence for a first guide RNA, a sequence for a second guide RNA, a first RNA template for a first homology arm DNA, and a second RNA template for a second homology arm DNA.

18. The method of claim 17, wherein the first homology arm DNA and the second homology arm DNA are separately complementary to distinct sites of a target genomic site.

19. The method of claim 17, wherein the editing retron (editron) further comprises an RNA template for a donor DNA that can be reverse transcribed by a reverse transcriptase.

20. The method of claim 19, wherein the reverse transcribed donor DNA is single stranded.

21. The method of claim 17, wherein the first RNA template for a first homology arm DNA flanks one end of a template for a donor DNA and the second RNA template for a second homology arm DNA flanks the second end of the template for the donor DNA.

22. The method of claim 19, wherein the first guide RNA and the second guide RNA bind to a target strand that is replaced by the reverse transcribed donor DNA.

23. The method of claim 19, wherein the RNA template for a donor DNA comprises an initiation site for a reverse transcriptase.

24. The method of claim 17, which provides at least 2-fold, at least 3-fold, at least 4-fold, at least 5-fold, at least 7-fold, at least 10-fold, at least 15-fold, at least 20-fold, at least 25-fold, at least 30-fold, at least 40-fold, at least 50-fold, at least 60-fold, at least 70-fold, at least 80-fold, at least 90-fold, at least 100-fold, or at least 200-fold more genomic deletions, genomic replacements, and/or genomic insertions compared to use of a single cutter guide RNA.
